(19) United States
(12) Patent Application Publication
(10) Pub. No.: US 2022/0349893 A1
(43) Pub. Date: Nov. 3, 2022

(54) METHODS FOR THE TREATMENT OF ARID1A-DEFICIENT CANCERS

(71) Applicant: The Wistar Institute of Anatomy and Biology, Philadelphia, PA (US)

(72) Inventors: Rugang ZHANG, Philadelphia, PA (US); Shuai WU, Philadelphia, PA (US)

(73) Assignee: The Wistar Institute of Anatomy and Biology, Philadelphia, PA (US)

(21) Appl. No.: 17/642,512

(22) PCT Filed: Sep. 11, 2020
(86) PCT No.: PCT/US2020/050488

§ 371 Date: Mar. 11, 2022

Related U.S. Application Data
(60) Provisional application No. 62/899,457, filed on Sep. 12, 2019.

(51) Int. Cl. G01N 33/574; A61P 35/00; A61K 31/501; A61K 31/155

(52) U.S. Cl. CPC G01N 33/57496; A61P 35/00; A61K 31/501; A61K 31/155; G01N 2333/4703

(57) ABSTRACT

The present disclosure is directed to the use of inhibitors of glutamate metabolism to treat cancers that have mutations in ARID1A. Thus, in accordance with the present disclosure, there is provided a method of treating a subject determined to have an ARIDIA-mutated cancer, pre-cancer or benign tumor comprising administering to said subject at least one inhibitor of glutamate metabolism.

### PRIORITY CLAIM

The present application claims benefit of priority to U.S. Provisional Application Ser. No. 62/899,457, filed Sep. 12, 2019, the entire contents of which are hereby incorporated by reference.

### FEDERAL FUNDING DISCLOSURE

The invention was made with government support under grant CA010815 awarded by the National Institutes of Health. The government has certain rights in the invention.

### BACKGROUND

### 1. Field

The present disclosure relates generally to the fields of medicine, oncology and genetics. More particularly, it methods of treating cancers, particularly those have ARIDIA mutations.

### 2. Related Art

SWI/SNF chromatin-remodeling complex is a major epigenetic regulator of gene expression through affecting the chromatin accessibility. This complex controls a large number of genes involving in development, lineage differentiation, cell cycle regulation DNA damage and repair and so on. SWI/SNF complex is made up of 15 subunits and about 20% of human cancers harbor mutations of specific subunits. Most mutations are loss-of-function mutation, indicating the important tumor suppressive role of SWI/SNF in tumorigenesis. Intriguingly, ARID1A mutation is prevalent in a wild variety of cancer types, for example, ˜57% mutation in ovarian clear cell carcinoma and ˜30% in renal clear cell carcinoma and endometrial carcinoma, and ARIDIA mutation correlates with poor prognosis in several types of cancers. However, there are limited effective therapies to target this mutation.

Majority of cancer cells experience metabolic reprogramming in order to keep the energy and intermediate metabolites demands for fasting proliferation and redox balance. Cancer cells consume more glucose through aerobic glycolysis compared with normal cells even in normoxic conditions, which is first observed by Otto Warburg. Several recent studies have shown that glutamine is another major nutrient for proliferative cells. Glutamine can be converted into and glutamate and subsequently converted into alpha-ketoglutarate (αKG) that replenish the tricarboxylic acid (TCA) cycle. Compared with glucose, glutamine not only provides a carbon source for biosynthesis, but also acts as a nitrogen source for the synthesis of nucleotide and other amino acids. In addition, because of high oxidative metabolism, most cancer cells generate excessive reactive oxygen species (ROS), which results in increased oxidative stress. One of the important antioxidants is the reduced form of glutathione (GSH). Glutamine-derived GSH can reduce the oxidative stress by scavenging the ROS in the cancer cells. However, because of the heterogeneity of tumor and microenvironment, cancer cells reprogram to use different metabolic pathways to adapt the needs for survival. This metabolic reprograming is driven by, but not limited to, oncogenes or tumor suppressors, such as TP53, MYC and RAS. Nutrients availability and microenvironment also can determine the metabolic preference. Therefore, understanding the heterogeneity of metabolic dependency enable us to develop therapeutic strategies to target tumor metabolism. Recent studies demonstrate SWI/SNF complex is required for metabolic homeostasis in liver and heart, but the biological consequence of SWI/SNF subunits mutation on tumor metabolism and how to utilize metabolism to target SWI/SNF mutated cancers are not well addressed.

### SUMMARY

Thus, in accordance with the present disclosure, there is provided a method of treating a subject determined to have an ARID1A-mutated cancer, pre-cancer or benign tumor comprising administering to said subject at least one inhibitor of glutamate metabolism. The at least one inhibitor of glutamate metabolism may be telaglenastat, diazooxonorleucine, OP-329 and/or OP-330. The method may further comprise treating said subject with a second cancer therapy, such as an inhibitor of aspartate biosynthesis, in particular metformin. The second cancer therapy may be chemotherapy, radiotherapy, immunotherapy (e.g., checkpoint inhibitor), hormonal therapy, toxin therapy or surgery. The checkpoint inhibitor may be a PD-1 inhibitor, a PD-L1 inhibitor or a CTLA-4 inhibitor. The checkpoint inhibitor may be an anti-PD-1 antibody (e.g., pembrolizumab; nivolumab, cemiplimab), an anti-PD-L1 antibody (e.g., atezolizumab, avelumab, durvalumab) or an anti-CTLA-4 antibody (ipilumumab).

The method may further comprise determining, prior to treating, that said subject has an ARID1A-mutated cancer, pre-cancer or benign tumor. Determining may comprise (a) obtaining a sample from said subject that contains protein and/or nucleic acids; and (b) determining mutation status of an ARID1A protein or nucleic acid encoding ARID1A comprising the sequence of SEQ ID NO: 1 in said sample. Determining may comprise a nucleic acid-based assay or a protein-based assay. The biological sample may be a fluid sample, such as blood, serum plasma, sputum, saliva, urine or nipple aspirate. The biological sample may be a tissue sample, such as a cancer, pre-cancer or benign tumor tissue sample.

The cancer may be breast cancer, pancreatic cancer, gastric cancer, or ovarian cancer, such as ovarian clear cell carcinoma. The subject may be a human subject or a non-human primate. The subject may have previously been diagnosed with cancer, a pre-cancer or a benign tumor. The cancer may be recurrent, primary, metastatic or multi-drug resistant. The inhibitor of glutamate metabolism may be administered more than once, such as daily, every other day, weekly, monthly and/or on a chronic basis.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.” The word “about” means plus or minus 5% of the stated number.

Other objects, features and advantages of the present disclosure will become apparent from the following detailed description. It should be understood, however, that the detailed description and the specific examples, while indicating specific embodiments of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1. ARID1A cells are dependent on glutamine metabolism. To explore the potential role of ARID1A in regulating metabolic reprogramming, the inventors knocked out ARID1A in ARID1A wild-type RMG1 and OVCA429 OCCC cells to mimic loss of ARID1A protein expression caused by >906% of ARID1A mutations. Compared with ARID1A wild-type controls, the glutamate metabolism/ammonia recycling pathway was significantly enriched by ARID1A knockout in RMG1 cells.

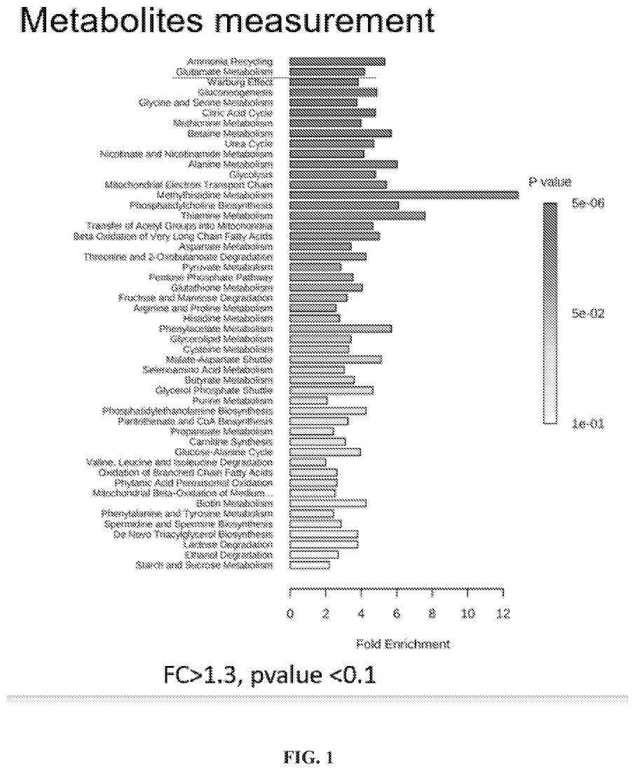

FIG. 2. ARID1A-deficient cells have high Gln dependence. Compared with ARID1A wild-type cells, ARID1A knockout cells significantly exacerbated the growth suppression induced by glutamine deprivation. Notably, glucose uptake was decreased by ARID1A knockout, which correlates with a decrease in sensitivity to glucose deprivation. Consistently, contribution of glutamine to oxygen consumption was significantly increased by ARID1A knockout as determined by Seahorse analysis, which was rescued by restoration of ARID1A expression in these cells.

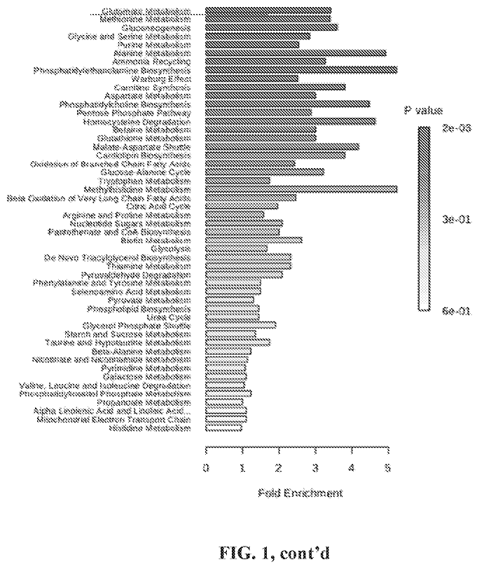

FIG. 3. ARID1A-deficient cells are sensitive to glutaminase inhibition. The inventors also tested CB-839, a specific glutaminase inhibition, in ARID1A wild-type control and knockout RMG1 cells. The inventors chose CB-839 for our experiments because it is the only GLS inhibitor that is now in clinical trials for other diseases and is proven safe in clinical trials including in combination studies. Indeed, compared with ARID1A wild-type control cells, ARID1A knockout significantly decreased the IC₅₀ of CB-839 in RMG1 cells by more than 300-fold. The observed effects are ARID1A dependent because the decrease in CB-839 IC₅₀ can be rescued by ectopic expression of wild-type ARID1A.

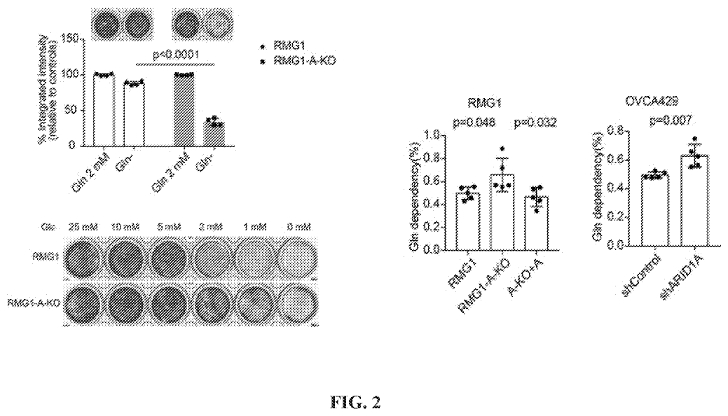

FIG. 4. ARID1A loss changes the response to CB-839. The inventors sought to determine how the ARID1A status differentially affects glutamine utilization. Toward this goal, they performed liquid chromatography and mass-spectral (LC-MS)/MS based analysis of metabolites in ARID1A wild-type control and knockout RMG1 OCCC cells with or without GLS inhibition by CB-839. Metabolic profiling revealed that ARID1A inactivation increases glutamine utilization by the TCA cycle and the use of glutamine to support aspartate and nucleotide biosynthesis. Pathway analysis revealed malate-aspartate shuttle as the top pathway enriched based on the differential response to CB-839 between ARID1A knockout cells and controls.

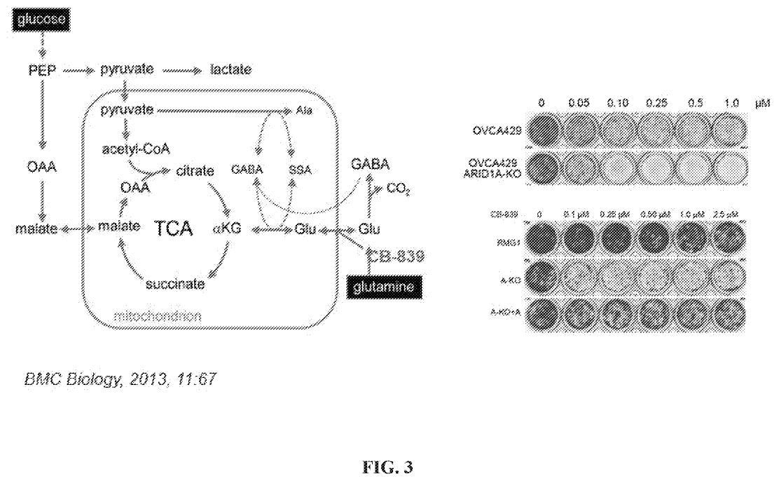

FIG. 5. Supplementation of aspartate abolishes sensitivity to CB-839. To examine whether ARID1A inactivation promotes aspartate and nucleotide synthesis from glutamine through the TCA cycle, addition of aspartate in the culture medium of ARID1A-mutated or knockout cells reduced the sensitivity to CB-839. In addition, ectopic expression of aspartate transporter SLC1A3 in RMG1 knockout cells that do not express endogenous SLC1A3 reduced the sensitivity to CB-839, further supporting that the observed effects are due to changes in aspartate.

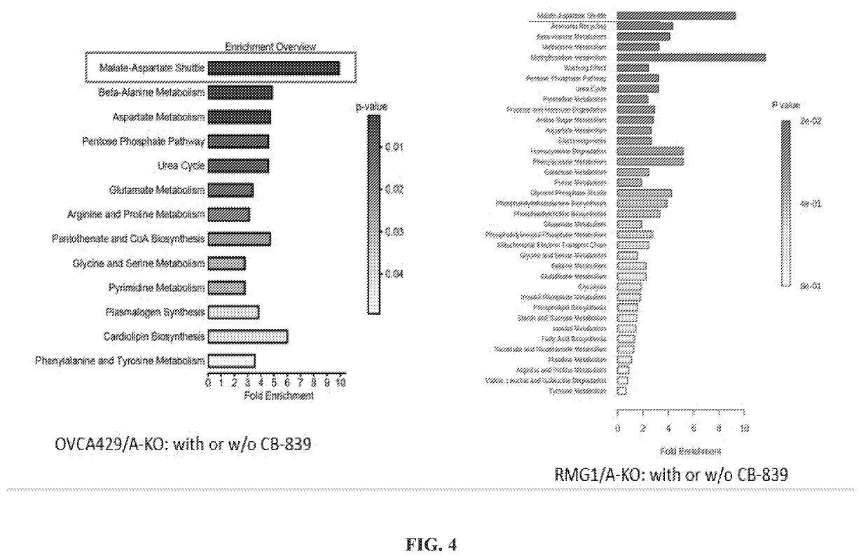

FIG. 6. Glutamine metabolism-related gene expression changes in ARID1A knock-out cells. Consistent with the RNA-seg results and further highlighting the role of ARID1A regulated GLS1 in the observed changes in glutamine metabolism, GLS1 is the top significantly upregulated gene that encodes an enzyme that can positively regulate the metabolism of glutamine into aspartate. Together, the inventors conclude that ARID1A inactivation creates glutamine dependence through both GLS1 upregulation and glutamine utilization such as aspartate generation and nucleotide synthesis.

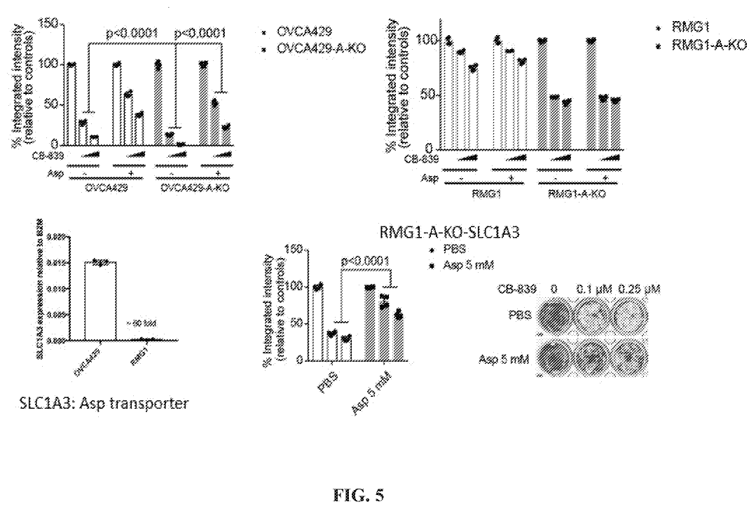

FIG. 7. Glutamine tracing assay shows high glutamine metabolism in ARID1A knock-out cells. Bars have the same order as key from top to bottom. The inventors performed liquid chromatography and mass-spectral (LC-MS)/MS based analysis of metabolites in ARID1A wild-type control and knockout RMG1 OCCC cells with or without GLS inhibition by CB-839. Cells were next incubated with ¹³C₅-glutamine to infer glutamine metabolism and associated metabolic pathways. The ¹³C₅-glutamine stable isotope tracer analysis revealed that ARID1A knockout increased the metabolism through glutamate, TCA cycle metabolites (such as α-ketoglutarate and citrate), aspartate and nucleotides (such as UMP). This suggests that in addition to increasing glutamine uptake by upregulating GLS1, ARID1A inactivation also increased the utilization of key glutamine metabolism metabolites such as aspartate to support the growth of ARID1A inactivated cells.

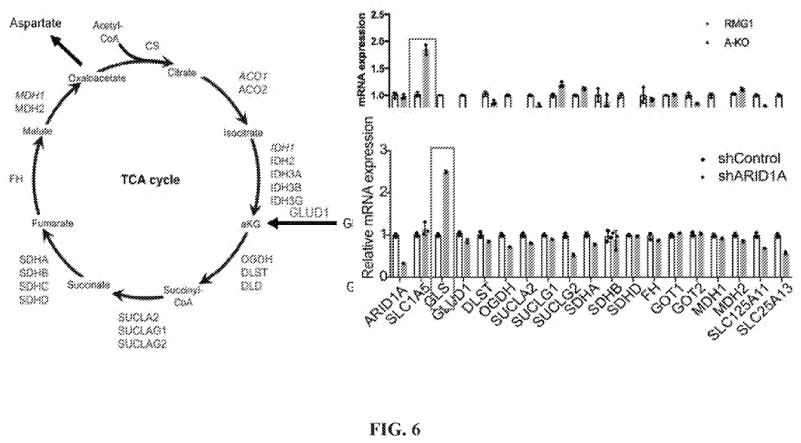

FIG. 8. GLS is a direct target of the SWI/SNF complex. To determine the mechanism underlying the observed glutamine dependence by ARID1A inactivation, the inventors cross-referenced ARID1A chromatin immunoprecipitation followed by next generation sequencing (ChIP-seq) analysis in ARID1A wild-type RMG1 cells with differentially expressed genes based on RNA-seq analysis in ARID1A wild-type control and knockout RMG1 cells. The analysis revealed GLSV as the top direct ARID1A target gene that was significantly upregulated by ARID1A knockout in glutamine metabolic pathway. Consistently, GLS1 is also a target of SNF5, a core subunit of the SWI/SNF complex and ARID1A knockout increased the association of RNA polymerase II (Pol II)'s association with the GLS1 promoter in RMG1 cells. Since repression of GLS1 by ARID1A correlates with changes in the SWI/SNF complex in the GLS1 promoter, the inventors examined whether inactivation of other SWI/SNF components will have similar effects on GLS1 expression. Indeed, knockdown of ARID1B or SNF5 subunits of the SWI/SNF complex also upregulated GLS1 expression and glutaminase inhibitor sensitivity.

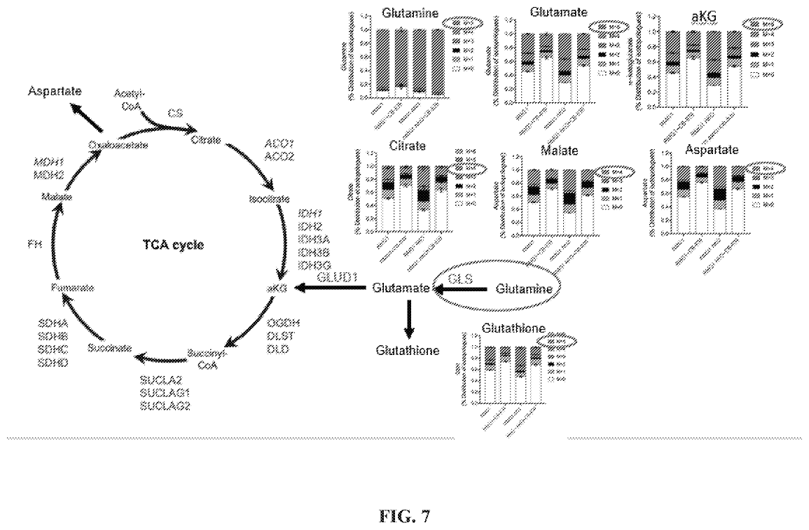

FIG. 9. A GLS inhibitor selectively represses cell proliferation in ARID1A-deficient cells. Bars have the same order as key from top to bottom. Isotype tracing liquid chromatography and mass-spectral (LC-MS)/MS based analysis of metabolites revealed that ARID1A knockout increased the metabolism through glutamate, TCA cycle metabolites (such as α-ketoglutarate and citrate), aspartate and nucleotides (such as UMP). Consistently, glutaminase inhibitor caused cell cycle arrest, but not apoptosis in ARID1A knockout cells

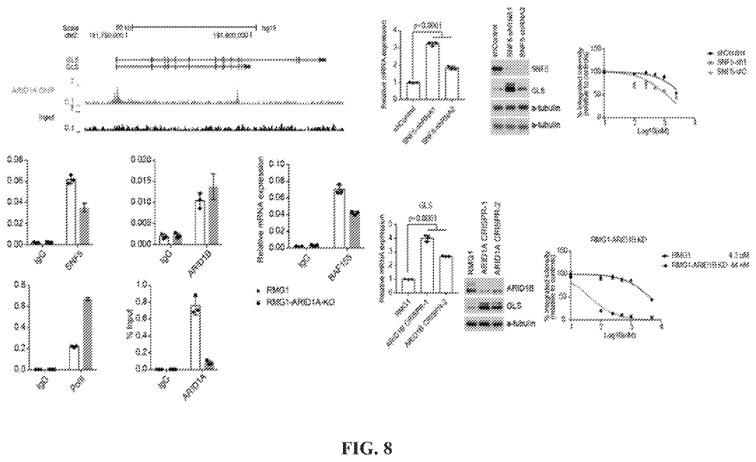

FIG. 10. A GLS inhibitor selectively decreases ARID1A-deficient tumors in vivo. The inventors sought to determine the therapeutic potential of GLS inhibitor CB-839 for ARID1A-mutated tumors. Toward this goal, they used three different mouse models. First, they used orthotopic xenograft models formed by ARID1A mutated TOV21G OCCC cells. Briefly, the orthotopically transplanted cells were allowed to grow for one week to establish the orthotopic tumors. Mice were then randomized and treated twice daily for three weeks with vehicle control or CB-839 (200 mg/kg) orally, feeding with normal and aspartic acid free food. The used tumor weight as a surrogate for tumor burden. Notably, the CB-839 treatment significantly reduced the burden of orthotopic xenografts formed by ARID1A mutated cells. The effect was enhanced when the mice were fed with aspartic acid free food. This correlated with a significant improvement of survival of tumor bearing mice

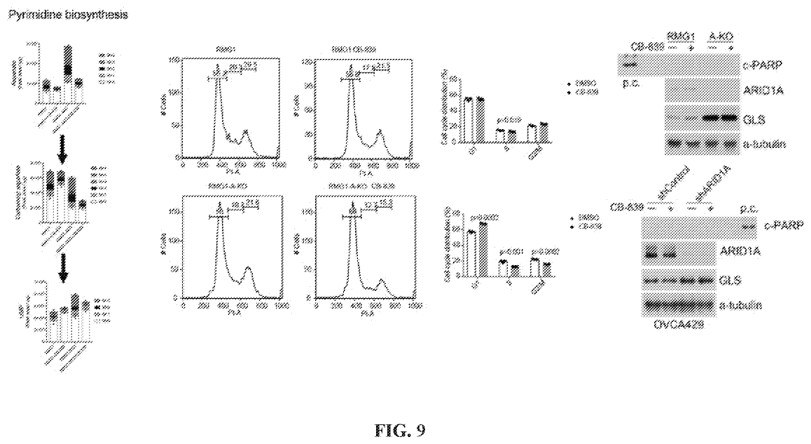

FIG. 11. A GLS inhibitor selectively decreases ARID1A-deficient tumors in vivo. Glutaminase inhibitor CB-839 treatment significantly reduced the burden of orthotopic xenografts formed by ARID1A knockout RMG1 cells. In contrast, CB-839 did not significantly affect the growth of tumors formed by ARID1A wild-type control RMG1 cells.

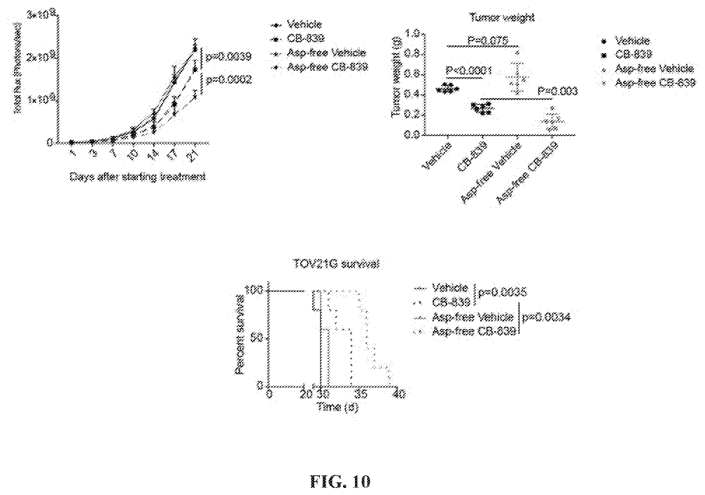

FIG. 12. ARID1A expression and SWI/SNF mutation status correlate with GLS expression. ARID1A expression was negative correlated with GLS expression in the cell line databased CCII. Since OCCC was not included in The Cancer Genome Atlas database, the inventors explored the correlation between GLS1 expression and mutations in the SWI/SNF complex in lung adenocarcinoma, renal clear cell carcinoma, skin cutaneous melanoma and uterine corpus endometrial carcinoma in which high frequency of mutations in the SWI/SNF subunits are observed. Indeed, GLS1 is expressed at a significantly higher levels in TP53 wild-type tumors with mutations in the SWI/SNF complex.

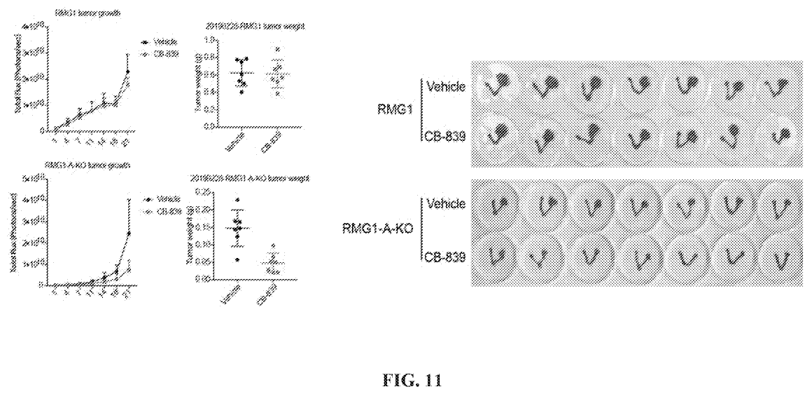

FIG. 13. ARID1A expression and SWI/SNF mutation status correlate with GLS expression. Bars have the same order as key from top to bottom. Tissue microarray was used to determine the correlation between ARID1A and GLS. The staining was defined to positive and negative based on the intensity and pathology of spot. GLS staining intensity was scored according to four grades: 0 (no staining), 1 (weak staining), 2 (moderate staining) and 3 (strong staining). The final score of each spot was determine by the percentage and intensity of staining.

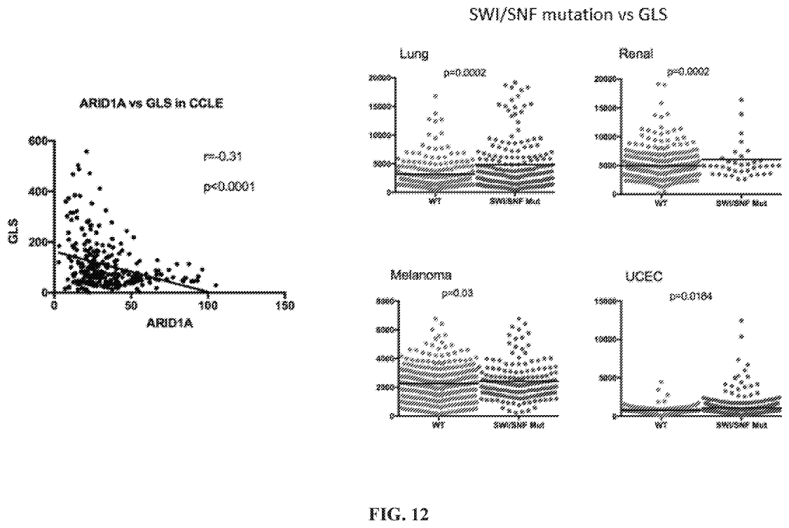

FIG. 14. A GLS inhibitor selectively decreases ARID1A-deficient PDX. ARID1A wild-type and mutated PDX OCCC xenograft models were used to evaluated the antitumor effect of glutaminase inhibitor. The result showed CB-839 significantly reduced the tumor burden in ARID1-mutated, but not ARID1A wild-type, OCCC PDXs.

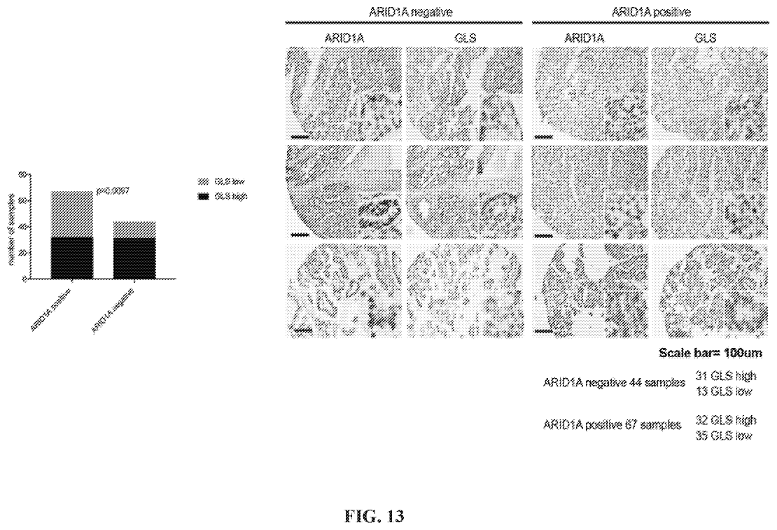

FIG. 15. Combination of GLSi and anti-PD-L1 in ARID1A knock out transgenic mice. Conditional genetic Arid1a^{flox/flox}/Pik3ca^{H1047R} OCCC mouse model was used. Consistent with the inventors' findings from orthotopic xenograft and PDX models, CB-839 treatment significantly reduced the burden in the pre-established genetic OCCC model. ARID124 mutation confers sensitivity to immune checkpoint blockades such as anti-PDL1. Thus, the inventors examined whether CB-839 synergizes with anti-PDL1 in the Arid1a/Pik3ca immune competent OCCC genetic mouse model. Indeed, a combination of CB-839 and anti-PDL1 was significantly more effective in reducing the tumor burden. Aspartic acid free food enhanced the antitumor effect of CB-839.

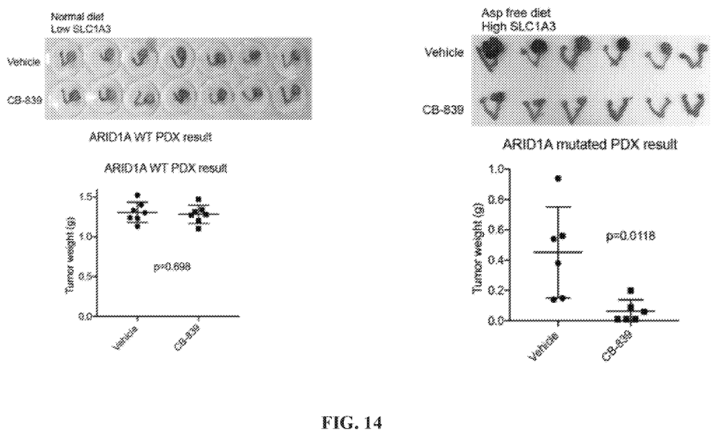

FIG. 16. GLSi increases the Gln levels in tumors. CB-839 treatment significantly increased glutamine levels in the treated tumors due to inhibition of glutamine utilization by the tumors.

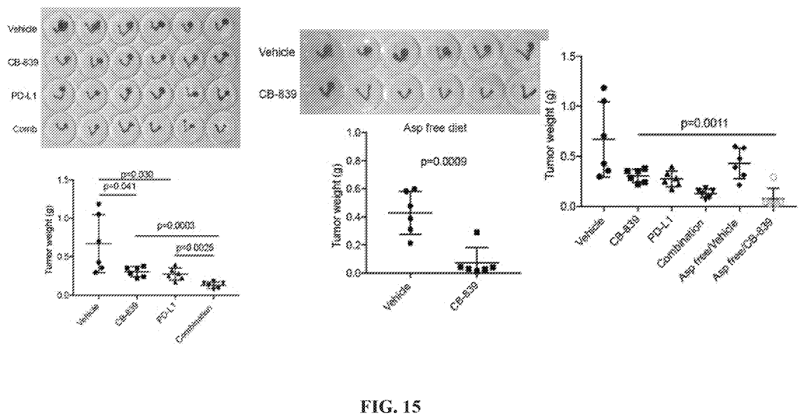

FIG. 17. After completing treatment, the transgenic mice were followed for survival, and the Kaplan-Meier survival curves for each of the indicated groups are shown (n=7 mice/group. Combination of CB-839 and anti-PDL1 significantly improved survival of tumor-bearing mice compared with either one of the individual treatments.

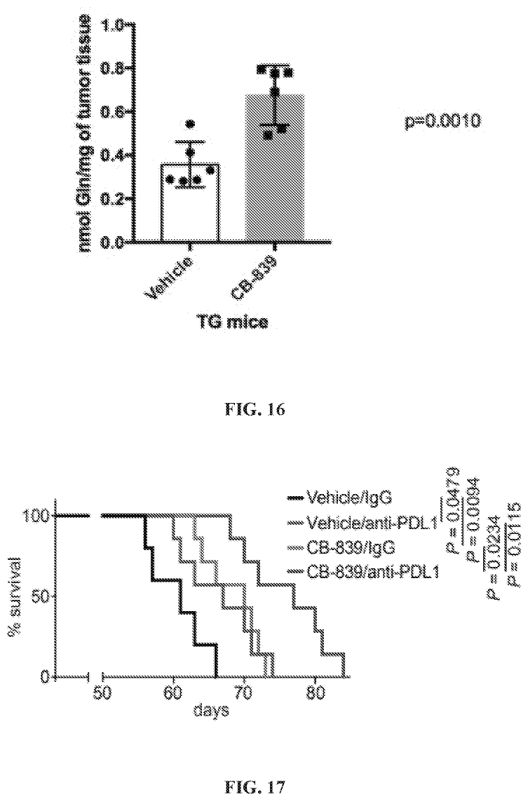

FIG. 18. CB-839 treatment prevented CD8 T cell exhaustion induced by anti-PDL1 antibody as evidenced by a decrease in PD-1 position CD8 T cells.

FIGS. 19A-B. CB-839 treatment. (FIG. 19A) At end of treatment, percentage of PD1 positive CD8 T cells was assessed by flow cytometry in the peritoneal wash in which tumors have disseminated. Flow cytometry gating strategy showing how the immune cells were profiled. (FIG. 19B) CB-839 did not affect PDL1 expression on ARID1A-mutated TOV21G cells in vitro.

### DESCRIPTION OF ILLUSTRATIVE EMBODIMENTS

The inventors have now shown that loss of ARID1A increases the glutamine dependency and sensitivity to glutaminase inhibitor in cancer cells. Upregulation of GLS by loss of ARID1A drives the glutamine flux into TCA cycle. The SWI/SNF complex directly represses GLS1 transcription and determines the sensitivity to GLS inhibitors. Insufficient of aspartate for DNA synthesis upon GLS inhibition causes cell cycle arrest in ARID1A-deficient cells. These data demonstrate that targeting glutamine metabolism is a promising therapeutic strategy in ARID1A-deficient cancers.

These and other aspects of the disclosure are described in greater detail below.

### I. SWI/SNF

In molecular biology, SWI/SNF (SWItch/Sucrose Non-Fermentable),¹ is a nucleosome remodeling complex found in eukaryotes. In simpler terms, it is a group of proteins that associate to remodel the way DNA is packaged. It is composed of several proteins—products of the SWI and SNF genes (SWI1, SWI2/SNF2, SWI3, SWI5, SWI6) as well as other polypeptides. It possesses a DNA-stimulated ATPase activity and can destabilise histone-DNA interactions in reconstituted nucleosomes in an ATP-dependent manner, though the exact nature of this structural change is unknown.

The human analogs of SWI/SNF are BAF (SWI/SNF-A) and PBAF (SWI/SNF-B). BAF in turn stands for “BRG1- or HBRM-associated factors” and PBAF is for “polybromo-associated BAF.”

It has been found that the SWI/SNF complex (in yeast) is capable of altering the position of nucleosomes along DNA. Two mechanisms for nucleosome remodeling by SWI/SNF have been proposed. The first model contends that a unidirectional diffusion of a twist defect within the nucleosomal DNA results in a corkscrew-like propagation of DNA over the octamer surface that initiates at the DNA entry site of the nucleosome. The other is known as the “bulge” or “loop-recapture” mechanism and it involves the dissociation of DNA at the edge of the nucleosome with reassociation of DNA inside the nucleosome, forming a DNA bulge on the octamer surface. The DNA loop would then propagate across the surface of the histone octamer in a wave-like manner, resulting in the repositioning of DNA without changes in the total number of histone-DNA contacts. A recent study has provided strong evidence against the twist diffusion mechanism and has further strengthened the loop-recapture model.

The mammalian SWI/SNF (mSWI/SNF) complex functions as a tumor suppressor in many human malignancies. Early studies identified that SWI/SNF subunits were frequently absent in cancer cell lines. It was first identified in 1998 as a tumor suppressor in rhabdoid tumors, a rare pediatric malignancy. As DNA sequencing costs diminished, many tumors were sequenced for the first time around 2010. Several of these studies revealed SWI/SNF to be a tumor suppressor in a number of diverse malignancies. Several studies revealed that subunits of the mammalian complex, including ARID1A, PBRM1, SMARCB1, SMARCA4, and ARID2, are frequently mutated in human cancers. A meta-analysis of many sequencing studies demonstrated SWI/SNF to be mutated in approximately 20% of human malignancies.

Electron microscopy studies of SWI/SNF and RSC (SWI/SNF-B) reveal large, lobed 1.1-1.3 MDa structures. No atomic-resolution structures of the entire SWI/SNF complex have been obtained to date, due to the protein complex being highly dynamic and composed of many subunits. However, domains and several individual subunits from yeast and mammals have been described. In particular, the cryo-EM structure of the ATPase Snf2 in complex with a nucleosome shows that nucleosomal DNA is locally deformed at the site of binding. A model of the mammalian ATPase SMARCA4 shows similar features, based on the high degree of sequence homology with yeast Snf2. The interface between two subunits, BAF155 (SMARCC1) and BAF47 (SMARCB1) was also resolved, providing important insights into the mechanisms of the SWI/SNF complex assembly pathway.

### II. ARID1A

AT-rich interactive domain-containing protein 1A (ARID1A) is a protein that in humans is encoded by the ARID1A gene. ARID1A has been shown to interact with SMARCB1 and SMARCA4.

ARID1A is a member of the SWI/SNF family, whose members have helicase and ATPase activities and are thought to regulate transcription of certain genes by altering the chromatin structure around those genes. The encoded protein is part of the large ATP-dependent chromatin remodelling complex SWI/SNF, which is required for transcriptional activation of genes normally repressed by chromatin. It possesses at least two conserved domains that could be important for its function. First, it has an ARID domain, which is a DNA-binding domain that can specifically bind an AT-rich DNA sequence known to be recognized by a SWI/SNF complex at the beta-globin locus. Second, the C-terminus of the protein can stimulate glucocorticoid receptor-dependent transcriptional activation. It is thought that the protein encoded by this gene confers specificity to the SWI/SNF complex and may recruit the complex to its targets through either protein-DNA or protein-protein interactions. Two transcript variants encoding different isoforms have been found for this gene.

This gene has been commonly found mutated in gastric cancers, ovarian clear cell carcinoma, and pancreatic cancer. In breast cancer distant metastases acquire inactivation mutations in ARID1A not seen in the primary tumor, and reduced ARID1A expression confers resistance to different drugs such as trastuzumab and mTOR inhibitors. these findings provide a rationale for why tumors accumulate ARID1A mutations.

An examplary protein accession number for human ARID1A is NP_006006. An exemplary mRNA accession number for human ARID1A is NM_139135.

### III. INHIBITORS

As discussed herein, inhibitors of glutamate metabolism may be advantageously applied to the treatment of cancer cells having mutated ARID1A. Inhibitors may be used individually or in combination. The inhibitors may inhibit and enzyme that participates in synthesis of glutamate in cancer cells or may otherwise impair the utilization of glutamate by cancer cells. The following are non-limiting examples of inhibitors according to the present disclosure.

A. Telaglenastat

Telaglenastat (CB-839) is a potent, selective, and orally bioavailable inhibitor of glutaminase (KGA and GAC) with IC₅₀'s of 28 and 23 nM for glutaminase in kidney and brain. Telaglenastat (0.1-1000 nM; 72 hours) has antiproliferative activity in HCC1806 and MDA-MB-231 cells with IC_{50S} of 49 nM and 26 nM, respectively. Telaglenastat (1 μM; 72 hours) activates caspase 3/7 and induces apoptosis in MDA-MB-231 and HCCC1806 cells. Telaglenastat significantly improves GAC and KGA expression levels in the majority of triple-negative breast cancer (TNBC) cell lines. The structure is shown below:

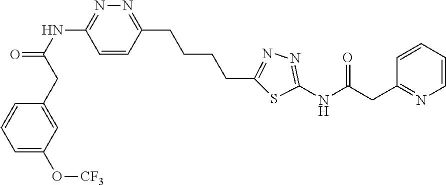 [SMILES: O=C(Cc1cccc(OC(F)(F)F)c1)Nc1ccc(CCCCc2nnc(NC(=O)Cc3ccccn3)s2)nn1]

B. Diazooxonorleucine

6-Diazo-5-oxo-L-norleucine (DON) is a glutamine antagonist, which was isolated originally from Streptomyces in a sample of Peruvian soil. It is a non-standard amino acid. The diazo compound was characterized in 1956 by Henry W. Dion and colleagues who suggested a possible use in cancer therapy. This antitumoral efficacy was confirmed in different animal models. DON was tested as chemotherapeutic agent in different clinical studies but was never approved. The last clinical results were published in 2008, though not as DON monotherapy but in combination with a recombinant glutaminase. In 2019, DON was shown to kill tumor cells while reversing disease symptoms and improve overall survival in late-stage experimental glioblastoma in mice, when combined with calorie-restricted ketogenic diet.

DON is a water-soluble yellowish powder, which can be dissolved also in aqueous solutions of methanol, acetone or ethanol, but dissolution in absolute alcohols is difficult. Solutions of at least 50 μM DON in 0.9% NaCl are lightly yellowish. The crystalline form appears as yellowish greenish needles. The specific rotation is [α]₂₆^{D}+21° (c=5.4% in H₂O). In phosphate buffer, pH 7 are the ultraviolet absorption maxima at 274 nm (E1%1 cm. 683) and 244 nm (E1%1 cm 376).

DON is used as inhibitor of different glutamine utilizing enzymes. Due to its similarity to glutamine it can enter catalytic centres of these enzymes and inhibits them by covalent binding, or more precisely by alkylation. DON targets include carbamoyl phosphatate synthase, CTP synthase, FGAR amidotransferase, guanosine monophosphate synthetase, PRPP amidotransferase, mitochondrial glutaminase, NAD synthase and asparagine synthetase. The structure of DON is shown below:

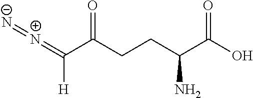 [SMILES: [N-]=[N+]=CC(=O)CC[C@H](N)C(=O)O]

C. Other Inhibitors OP-329

The disclosure contemplates other inhibitors of glutamate metabolism, such as the glutaminase inhibitors OP-329 and OP-330. Relevant disclosure from U.S. Patent Publication No. US20130109643 is hereby incorporated by reference.

### IV. TREATING CANCERS

A. Cancer

Cancer encompasses a group of diseases involving abnormal cell growth with the potential to invade or spread to other parts of the body. These contrast with benign tumors, which do not spread to other parts of the body. Possible signs and symptoms include a lump, abnormal bleeding, prolonged cough, unexplained weight loss and a change in bowel movements. While these symptoms may indicate cancer, they may have other causes. Over 100 types of cancers affect humans.

Cancer can spread from its original site by local spread, lymphatic spread to regional lymph nodes or by hematogenous spread via the blood to distant sites, known as metastasis. When cancer spreads by a hematogenous route, it usually spreads all over the body. The symptoms of metastatic cancers depend on the tumor location and can include enlarged lymph nodes (which can be felt or sometimes seen under the skin and are typically hard), enlarged liver or enlarged spleen, which can be felt in the abdomen, pain or fracture of affected bones and neurological symptoms.

Many treatment options for cancer exist. The primary ones include surgery, chemotherapy, radiation therapy, hormonal therapy, targeted therapy and palliative care. Which treatments are used depends on the type, location and grade of the cancer as well as the patient's health and personal wishes. The treatment intent may or may not be curative.

The therapeutic methods of the disclosure in general include administration of a therapeutically effective amount of the compositions described herein to a subject in need thereof, including a mammal, particularly a human. Such treatment will be suitably administered to subjects, particularly humans, suffering from cancer or having a symptom thereof.

The cancer may be a carcinoma, sarcoma, lymphoma, leukemia, melanoma, mesothelioma, multiple myeloma, or seminoma. In some embodiments, the cancer is of the bladder, blood, bone, brain, breast, central nervous system, cervix, colon, endometrium, esophagus, gall bladder, gastrointestinal tract, genitalia, genitourinary tract, head, kidney, larynx, liver, lung, muscle tissue, neck, oral or nasal mucosa, ovary, pancreas, prostate, skin, spleen, small intestine, large intestine, stomach, testicle, or thyroid. In some embodiments, the cancer is ovarian cancer, pancreatic cancer, breast cancer, or gastric cancer.

B. Theranostic Methods

In one embodiment, the disclosure provides methods to assess the ARID1A status of a cancer being treated. The method includes the step of determining whether a cancer patient's cancer has a mutated ARID1A gene or protein prior to administering a therapeutic composition as described herein. The analysis is useful in predicting whether the subject will respond to a glutamate metabolism inhibitor—if so, then the glutamate inhibitor is administered, and if not, then another therapy is employed. The following exemplary techniques can be employed to examine ARID1A status.

1. Nucleic Acid-Based Detection Methods

Nucleic acid-based detection methods may be employed to identify cancers with mutant ARID1A. The following is a discussion of such methods, which are applicable to assessing mutations in ARID1A.

In certain embodiments, the disclosure relates to methods of characterizing and treating cancer by detecting mutant ARID1A. Such cancers can be a breast cancer, lung cancer, colon cancer, pancreatic cancer, renal cancer, stomach cancer, liver cancer, bone cancer, hematological cancer (e.g., leukemia or lymphoma), neural tissue cancer, melanoma, ovarian cancer, testicular cancer, prostate cancer, cervical cancer, vaginal cancer, or bladder cancer cell. In addition, the methods of the disclosure can be applied to a wide range of species, e.g., humans, non-human primates (e.g., monkeys, baboons, or chimpanzees), horses, cattle, pigs, sheep, goats, dogs, cats, rabbits, guinea pigs, gerbils, hamsters, rats, and mice.

i. Hybridization

Methods looking at DNA or mRNA all fundamentally rely, at a basic level, on nucleic acid hybridization. Hybridization is defined as the ability of a nucleic acid to selectively form duplex molecules with complementary stretches of DNAs and/or RNAs. Depending on the application envisioned, one would employ varying conditions of hybridization to achieve varying degrees of selectivity of the probe or primers for the target sequence.

Typically, a probe or primer of between 13 and 100 nucleotides, preferably between 17 and 100 nucleotides in length up to 1-2 kilobases or more in length will allow the formation of a duplex molecule that is both stable and selective. Molecules having complementary sequences over contiguous stretches greater than 20 bases in length are generally preferred, to increase stability and selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

For certain applications, for example, lower stringency conditions may be used. Under these conditions, hybridization may occur even though the sequences of the hybridizing strands are not perfectly complementary but are mismatched at one or more positions. Conditions may be rendered less stringent by increasing salt concentration and/or decreasing temperature. For example, a medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results.

In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In certain embodiments, it will be advantageous to employ nucleic acids of defined sequences of the present invention in combination with an appropriate means, such as a label, for determining hybridization. A wide variety of appropriate indicator means are known in the art, including fluorescent, radioactive, enzymatic or other ligands, such as avidin/biotin, which are capable of being detected. In preferred embodiments, one may desire to employ a fluorescent label or an enzyme tag such as urease, alkaline phosphatase or peroxidase, instead of radioactive or other environmentally undesirable reagents. In the case of enzyme tags, colorimetric indicator substrates are known that can be employed to provide a detection means that is visibly or spectrophotometrically detectable, to identify specific hybridization with complementary nucleic acid containing samples.

In general, it is envisioned that the probes or primers described herein will be useful as reagents in solution hybridization, as in PCR™, for detection of expression of corresponding genes, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the Specification are incorporated herein by reference.

ii. Nucleic Acid Amplification

Since many mRNAs are present in relatively low abundance, nucleic acid amplification greatly enhances the ability to assess expression. The general concept is that nucleic acids can be amplified using paired primers flanking the region of interest. The term “primer,” as used herein, is meant to encompass any nucleic acid that is capable of priming the synthesis of a nascent nucleic acid in a template-dependent process. Typically, primers are oligonucleotides from ten to twenty and/or thirty base pairs in length, but longer sequences can be employed. Primers may be provided in double-stranded and/or single-stranded form, although the single-stranded form is preferred.

Pairs of primers designed to selectively hybridize to nucleic acids corresponding to selected genes are contacted with the template nucleic acid tinder conditions that permit selective hybridization. Depending upon the desired application, high stringency hybridization conditions may be selected that will only allow hybridization to sequences that are completely complementary to the primers. In other embodiments, hybridization may occur under reduced stringency to allow for amplification of nucleic acids contain one or more mismatches with the primer sequences. Once hybridized, the template-primer complex is contacted with one or more enzymes that facilitate template-dependent nucleic acid synthesis. Multiple rounds of amplification, also referred to as “cycles,” are conducted until a sufficient amount of amplification product is produced.

The amplification product may be detected or quantified. In certain applications, the detection may be performed by visual means. Alternatively, the detection may involve indirect identification of the product via chemilluminescence, radioactive scintigraphy of incorporated radiolabel or fluorescent label or even via a system using electrical and/or thermal impulse signals.

A number of template dependent processes are available to amplify the oligonucleotide sequences present in a given template sample. One of the best-known amplification methods is the polymerase chain reaction (referred to as PCR™) which is described in detail in U.S. Pat. Nos. 4,683,195, 4,683,202 and 4,800,159, and in Innis et al, 1988, each of which is incorporated herein by reference in their entirety.

A reverse transcriptase PCR™ amplification procedure may be performed to quantify the amount of mRNA amplified. Methods of reverse transcribing RNA into cDNA are well known (see Sambrook et al., 1989). Alternative methods for reverse transcription utilize thermostable DNA polymerases. These methods are described in WO 90/07641. Polymerase chain reaction methodologies are well known in the art. Representative methods of RT-PCR are described in U.S. Pat. No. 5,882,864.

Whereas standard PCR usually uses one pair of primers to amplify a specific sequence, multiplex-PCR (MPCR) uses multiple pairs of primers to amplify many sequences simultaneously. The presence of many PCR primers in a single tube could cause many problems, such as the increased formation of misprimed PCR products and “primer dimers,” the amplification discrimination of longer DNA fragment and so on. Normally, MPCR buffers contain a Taq Polymerase additive, which decreases the competition among amplicons and the amplification discrimination of longer DNA fragment during MPCR. MPCR products can further be hybridized with gene-specific probe for verification. Theoretically, one should be able to use as many as primers as necessary. However, due to side effects (primer dimers, misprimed PCR products, etc.) caused during MPCR, there is a limit (less than 20) to the number of primers that can be used in a MPCR reaction. See also European Application No. 0 364 255 and Mueller and Wold (1989).

Another method for amplification is ligase chain reaction (“LCR”), disclosed in European Application No. 320 308, incorporated herein by reference in its entirety U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2 202 328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence-based amplification (NASBA) and 3SR (Kwoh et al., 1989; Gingeras et al., PCT Application WO 88/10315, incorporated herein by reference in their entirety). European Application No. 329 822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) disclose a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1990; Ohara et al, 1989).

iii. Detection of Nucleic Acids

Following any amplification, it may be desirable to separate the amplification product from the template and/or the excess primer. In one embodiment, amplification products are separated by agarose, agarose-acrylamide or polyacrylamide gel electrophoresis using standard methods (Sambrook et al., 1989). Separated amplification products may be cut out and eluted from the gel for further manipulation. Using low melting point agarose gels, the separated band may be removed by heating the gel, followed by extraction of the nucleic acid.

Separation of nucleic acids may also be achieved by chromatographic techniques known in art. There are many kinds of chromatography which may be used in the practice of the present invention, including adsorption, partition, ion-exchange, hydroxylapatite, molecular sieve, reverse-phase, column, paper, thin-layer, and gas chromatography as well as HPLC.

In certain embodiments, the amplification products are visualized. A typical visualization method involves staining of a gel with ethidium bromide and visualization of bands under UV light. Alternatively, if the amplification products are integrally labeled with radio- or fluorometrically-labeled nucleotides, the separated amplification products can be exposed to x-ray film or visualized under the appropriate excitatory spectra.

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In particular embodiments, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

iv. Nucleic Acid Arrays

Microarrays comprise a plurality of polymeric molecules spatially distributed over, and stably associated with, the surface of a substantially planar substrate, e.g., biochips. Microarrays of polynucleotides have been developed and find use in a variety of applications, such as screening and DNA sequencing. One area in particular in which microarrays find use is in gene expression analysis.

In gene expression analysis with microarrays, an array of “probe” oligonucleotides is contacted with a nucleic acid sample of interest, i.e., target, such as polyA mRNA from a particular tissue type. Contact is carried out under hybridization conditions and unbound nucleic acid is then removed. The resultant pattern of hybridized nucleic acid provides information regarding the genetic profile of the sample tested. Methodologies of gene expression analysis on microarrays are capable of providing both qualitative and quantitative information.

A variety of different arrays which may be used are known in the art. The probe molecules of the arrays which are capable of sequence specific hybridization with target nucleic acid may be polynucleotides or hybridizing analogues or mimetics thereof, including: nucleic acids in which the phosphodiester linkage has been replaced with a substitute linkage, such as phophorothioate, methylimino, methylphosphonate, phosphoramidate, guanidine and the like; nucleic acids in which the ribose subunit has been substituted, e.g., hexose phosphodiester; peptide nucleic acids; and the like. The length of the probes will generally range from 10 to 1000 nts, where in some embodiments the probes will be oligonucleotides and usually range from 15 to 150 nts and more usually from 15 to 100 nts in length, and in other embodiments the probes will be longer, usually ranging in length from 150 to 1000 nts, where the polynucleotide probes may be single- or double-stranded, usually single-stranded, and may be PCR fragments amplified from cDNA.

The probe molecules on the surface of the substrates will correspond to selected genes being analyzed and be positioned on the array at a known location so that positive hybridization events may be correlated to expression of a particular gene in the physiological source from which the target nucleic acid sample is derived. The substrates with which the probe molecules are stably associated may be fabricated from a variety of materials, including plastics, ceramics, metals, gels, membranes, glasses, and the like. The arrays may be produced according to any convenient methodology, such as preforming the probes and then stably associating them with the surface of the support or growing the probes directly on the support. A number of different array configurations and methods for their production are known to those of skill in the art and disclosed in U.S. Pat. Nos. 5,445,934, 5,532,128, 5,556,752, 5,242,974, 5,384,261, 5,405,783, 5,412,087, 5,424,186, 5,429,807, 5,436,327, 5,472,672, 5,527,681, 5,529,756, 5,545,531, 5,554,501, 5,561,071, 5,571,639, 5,593,839, 5,599,695, 5,624,711, 5,658,734, 5,700,637, and 6,004,755.

Following hybridization, where non-hybridized labeled nucleic acid is capable of emitting a signal during the detection step, a washing step is employed where unhybridized labeled nucleic acid is removed from the support surface, generating a pattern of hybridized nucleic acid on the substrate surface. A variety of wash solutions and protocols for their use are known to those of skill in the art and may be used.

Where the label on the target nucleic acid is not directly detectable, one then contacts the array, now comprising bound target, with the other member(s) of the signal producing system that is being employed. For example, where the label on the target is biotin, one then contacts the array with streptavidin-fluorescer conjugate under conditions sufficient for binding between the specific binding member pairs to occur. Following contact, any unbound members of the signal producing system will then be removed, e.g., by washing. The specific wash conditions employed will necessarily depend on the specific nature of the signal producing system that is employed and will be known to those of skill in the art familiar with the particular signal producing system employed.

The resultant hybridization pattern(s) of labeled nucleic acids may be visualized or detected in a variety of ways, with the particular manner of detection being chosen based on the particular label of the nucleic acid, where representative detection means include scintillation counting, autoradiography, fluorescence measurement, calorimetric measurement, light emission measurement and the like.

Prior to detection or visualization, where one desires to reduce the potential for a mismatch hybridization event to generate a false positive signal on the pattern, the array of hybridized target/probe complexes may be treated with an endonuclease under conditions sufficient such that the endonuclease degrades single stranded, but not double stranded DNA. A variety of different endonucleases are known and may be used, where such nucleases include: mung bean nuclease, S1 nuclease, and the like. Where such treatment is employed in an assay in which the target nucleic acids are not labeled with a directly detectable label, e.g., in an assay with biotinylated target nucleic acids, the endonuclease treatment will generally be performed prior to contact of the array with the other member(s) of the signal producing system, e.g., fluorescent-streptavidin conjugate. Endonuclease treatment, as described above, ensures that only end-labeled target/probe complexes having a substantially complete hybridization at the 3′ end of the probe are detected in the hybridization pattern.

Following hybridization and any washing step(s) and/or subsequent treatments, as described above, the resultant hybridization pattern is detected. In detecting or visualizing the hybridization pattern, the intensity or signal value of the label will be not only be detected but quantified, by which is meant that the signal from each spot of the hybridization will be measured and compared to a unit value corresponding the signal emitted by known number of end-labeled target nucleic acids to obtain a count or absolute value of the copy number of each end-labeled target that is hybridized to a particular spot on the array in the hybridization pattern.

2. Protein-Based Detection Methods

i. Immunodetection

In still further embodiments, there are immunodetection methods for identifying and/or quantifying mutant ARID1A. These methods may, in certain embodiments, be applied to the treatment of cancer, such as those discussed above.

Some immunodetection methods include enzyme linked immunosorbent assay (ELISA), radioimmunoassay (RIA), immunoradiometric assay, fluoroimmunoassay, chemiluminescent assay, bioluminescent assay, and Western blot to mention a few. In particular, a competitive assay for the detection and quantitation of TSP1 antibodies also is provided. The steps of various useful immunodetection methods have been described in the scientific literature, such as, e.g., Doolittle and Ben-Zeev (1999), Gulbis and Galand (1993), De Jager et al. (1993), and Nakamura et al. (1987). In general, the immunobinding methods include obtaining a sample and contacting the sample with a first antibody in accordance with embodiments discussed herein, as the case may be, under conditions effective to allow the formation of immunocomplexes.

Contacting the chosen biological sample with the antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to ARID1A present. After this time, the sample-antibody composition, such as a tissue section, ELISA plate, dot blot or Western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. Patents concerning the use of such labels include U.S. Pat. Nos. 3,817,837, 3,850,752, 3,939,350, 3,996,345, 4,277,437, 4,275,149 and 4,366,241. Of course, one may find additional advantages through the use of a secondary binding ligand such as a second antibody and/or a biotin/avidin ligand binding arrangement, as is known in the art.

The antibody employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two-step approach. A second binding ligand, such as an antibody that has binding affinity for the antibody, is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

One method of immunodetection uses two different antibodies. A first biotinylated antibody is used to detect the target antigen, and a second antibody is then used to detect the biotin attached to the complexed biotin. In that method, the sample to be tested is first incubated in a solution containing the first step antibody. If the target antigen is present, some of the antibody binds to the antigen to form a biotinylated antibody/antigen complex. The antibody/antigen complex is then amplified by incubation in successive solutions of streptavidin (or avidin), biotinylated DNA, and/or complementary biotinylated DNA, with each step adding additional biotin sites to the antibody/antigen complex. The amplification steps are repeated until a suitable level of amplification is achieved, at which point the sample is incubated in a solution containing the second step antibody against biotin. This second step antibody is labeled, as for example with an enzyme that can be used to detect the presence of the antibody/antigen complex by histoenzymology using a chromogen substrate. With suitable amplification, a conjugate can be produced which is macroscopically visible.

Another known method of immunodetection takes advantage of the immuno-PCR (Polymerase Chain Reaction) methodology. The PCR method is similar to the Cantor method up to the incubation with biotinylated DNA, however, instead of using multiple rounds of streptavidin and biotinylated DNA incubation, the DNA/biotin/streptavidin/antibody complex is washed out with a low pH or high salt buffer that releases the antibody. The resulting wash solution is then used to carry out a PCR reaction with suitable primers with appropriate controls. At least in theory, the enormous amplification capability and specificity of PCR can be utilized to detect a single antigen molecule.

ii. ELISAs

Immunoassays, in their most simple sense, are binding assays. Certain preferred immunoassays are the various types of enzyme linked immunosorbent assays (ELISAs) and radioimmunoassays (RIA) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, the antibodies of the disclosure are immobilized onto a selected surface exhibiting protein affinity, such as a well in a polystyrene microtiter plate. Then, a test composition suspected of containing the TSP1 is added to the wells. After binding and washing to remove non-specifically bound immune complexes, the bound antigen may be detected. Detection may be achieved by the addition of another anti-ARID1A antibody that is linked to a detectable label. This type of ELISA is a simple “sandwich ELISA.” Detection may also be achieved by the addition of a second anti-ARID1A antibody, followed by the addition of a third antibody that has binding affinity for the second antibody, with the third antibody being linked to a detectable label.

In another exemplary ELISA, the samples suspected of containing the ARID1A are immobilized onto the well surface and then contacted with anti-ARID1A antibody. After binding and washing to remove non-specifically bound immune complexes, the bound anti-ARID1A antibodies are detected. Where the initial anti-ARID1A antibodies are linked to a detectable label, the immune complexes may be detected directly. Again, the immune complexes may be detected using a second antibody that has binding affinity for the first anti-ARID1A antibody, with the second antibody being linked to a detectable label.

Irrespective of the format employed, ELISAs have certain features in common, such as coating, incubating and binding, washing to remove non-specifically bound species, and detecting the bound immune complexes. These are described below.

In coating a plate with either antigen or antibody, one will generally incubate the wells of the plate with a solution of the antigen or antibody, either overnight or for a specified period of hours. The wells of the plate will then be washed to remove incompletely adsorbed material. Any remaining available surfaces of the wells are then “coated” with a nonspecific protein that is antigenically neutral with regard to the test antisera. These include bovine serum albumin (BSA), casein or solutions of milk powder. The coating allows for blocking of nonspecific adsorption sites on the immobilizing surface and thus reduces the background caused by nonspecific binding of antisera onto the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the biological sample to be tested under conditions effective to allow immune complex (antigen/antibody) formation. Detection of the immune complex then requires a labeled secondary binding ligand or antibody, and a secondary binding ligand or antibody in conjunction with a labeled tertiary antibody or a third binding ligand.

“Under conditions effective to allow immune complex (antigen/antibody) formation” means that the conditions preferably include diluting the antigens and/or antibodies with solutions such as BSA, bovine gamma globulin (BGG) or phosphate buffered saline (PBS)/Tween. These added agents also tend to assist in the reduction of nonspecific background.

The “suitable” conditions also mean that the incubation is at a temperature or for a period of time sufficient to allow effective binding. Incubation steps are typically from about 1 to 2 to 4 hours or so, at temperatures preferably on the order of 25° C. to 27° C. or may be overnight at about 4° C. or so.

Following all incubation steps in an ELISA, the contacted surface is washed so as to remove non-complexed material. A preferred washing procedure includes washing with a solution such as PBS/Tween, or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. Preferably, this will be an enzyme that will generate color development upon incubating with an appropriate chromogenic substrate. Thus, for example, one will desire to contact or incubate the first and second immune complex with a urease, glucose oxidase, alkaline phosphatase or hydrogen peroxidase-conjugated antibody for a period of time and under conditions that favor the development of further immune complex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea, or bromocresol purple, or 2,2′-azino-di-(3-ethyl-benzthiazoline-6-sulfonic acid (ABTS), or H₂O₂, in the case of peroxidase as the enzyme label. Quantification is then achieved by measuring the degree of color generated, e.g., using a visible spectra spectrophotometer.

iii. Western Blot

The Western blot (alternatively, protein immunoblot) is an analytical technique used to detect specific proteins in a given sample of tissue homogenate or extract. It uses gel electrophoresis to separate native or denatured proteins by the length of the polypeptide (denaturing conditions) or by the 3-D structure of the protein (native/non-denaturing conditions). The proteins are then transferred to a membrane (typically nitrocellulose or PVDF), where they are probed (detected) using antibodies specific to the target protein.

Samples may be taken from whole tissue or from cell culture. In most cases, solid tissues are first broken down mechanically using a blender (for larger sample volumes), using a homogenizer (smaller volumes), or by sonication. Cells may also be broken open by one of the above mechanical methods. However, it should be noted that bacteria, virus or environmental samples can be the source of protein and thus Western blotting is not restricted to cellular studies only. Assorted detergents, salts, and buffers may be employed to encourage lysis of cells and to solubilize proteins. Protease and phosphatase inhibitors are often added to prevent the digestion of the sample by its own enzymes. Tissue preparation is often done at cold temperatures to avoid protein denaturing.

The proteins of the sample are separated using gel electrophoresis. Separation of proteins may be by isoelectric point (pI), molecular weight, electric charge, or a combination of these factors. The nature of the separation depends on the treatment of the sample and the nature of the gel. This is a very useful way to determine a protein. It is also possible to use a two-dimensional (2-D) gel which spreads the proteins from a single sample out in two dimensions. Proteins are separated according to isoelectric point (pH at which they have neutral net charge) in the first dimension, and according to their molecular weight in the second dimension.

In order to make the proteins accessible to antibody detection, they are moved from within the gel onto a membrane made of nitrocellulose or polyvinylidene difluoride (PVDF). The membrane is placed on top of the gel, and a stack of filter papers placed on top of that. The entire stack is placed in a buffer solution which moves up the paper by capillary action, bringing the proteins with it. Another method for transferring the proteins is called electroblotting and uses an electric current to pull proteins from the gel into the PVDF or nitrocellulose membrane. The proteins move from within the gel onto the membrane while maintaining the organization they had within the gel. As a result of this blotting process, the proteins are exposed on a thin surface layer for detection (see below). Both varieties of membrane are chosen for their non-specific protein binding properties (i.e., binds all proteins equally well). Protein binding is based upon hydrophobic interactions, as well as charged interactions between the membrane and protein. Nitrocellulose membranes are cheaper than PVDF but are far more fragile and do not stand up well to repeated probings. The uniformity and overall effectiveness of transfer of protein from the gel to the membrane can be checked by staining the membrane with Coomassie Brilliant Blue or Ponceau S dyes. Once transferred, proteins are detected using labeled primary antibodies, or unlabeled primary antibodies followed by indirect detection using labeled protein A or secondary labeled antibodies binding to the Fc region of the primary antibodies.

iv. Immunohistochemistry

The antibodies may also be used in conjunction with both fresh-frozen and/or formalin-fixed, paraffin-embedded tissue blocks prepared for study by immunohistochemistry (IHC). The method of preparing tissue blocks from these particulate specimens has been successfully used in previous IHC studies of various prognostic factors and is well known to those of skill in the art (Brown et al., 1990; Abbondanzo et al., 1990; Allred et al., 1990).

Briefly, frozen-sections may be prepared by rehydrating 50 ng of frozen “pulverized” tissue at room temperature in phosphate buffered saline (PBS) in small plastic capsules; pelleting the particles by centrifugation; resuspending them in a viscous embedding medium (OCT); inverting the capsule and/or pelleting again by centrifugation; snap-freezing in −70° C. isopentane; cutting the plastic capsule and/or removing the frozen cylinder of tissue; securing the tissue cylinder on a cryostat microtome chuck; and/or cutting 25-50 serial sections from the capsule. Alternatively, whole frozen tissue samples may be used for serial section cuttings.

Permanent-sections may be prepared by a similar method involving rehydration of the 50 mg sample in a plastic microfuge tube; pelleting; resuspending in 10% formalin for 4 hours fixation; washing/pelleting; resuspending in warm 2.5% agar; pelleting; cooling in ice water to harden the agar; removing the tissue/agar block from the tube; infiltrating and/or embedding the block in paraffin; and/or cutting up to 50 serial permanent sections. Again, whole tissue samples may be substituted.

v. Immunodetection Kits

In still further embodiments, there are immunodetection kits for use with the immunodetection methods described above. The immunodetection kits will thus comprise, in suitable container means, a first antibody that binds to TSP1 antigen, and optionally an immunodetection reagent.

In certain embodiments, the TSP1 antibody may be pre-bound to a solid support, such as a column matrix and/or well of a microtitre plate. The immunodetection reagents of the kit may take any one of a variety of forms, including those detectable labels that are associated with or linked to the given antibody. Detectable labels that are associated with or attached to a secondary binding ligand are also contemplated. Exemplary secondary ligands are those secondary antibodies that have binding affinity for the first anti body.

Further suitable immunodetection reagents for use in the present kits include the two-component reagent that comprises a secondary antibody that has binding affinity for the first antibody, along with a third antibody that has binding affinity for the second antibody, the third antibody being linked to a detectable label. As noted above, a number of exemplary labels are known in the art and all such labels may be employed in connection with embodiments discussed herein.

The kits may further comprise a suitably aliquoted composition of the TSP1 antigen, whether labeled or unlabeled, as may be used to prepare a standard curve for a detection assay. The kits may contain antibody-label conjugates either in fully conjugated form, in the form of intermediates, or as separate moieties to be conjugated by the user of the kit. The components of the kits may be packaged either in aqueous media or in lyophilized form.

The container means of the kits will generally include at least one vial, test tube, flask, bottle, syringe or other container means, into which the antibody may be placed, or preferably, suitably aliquoted. The kits will also include a means for containing the antibody, antigen, and any other reagent containers in close confinement for commercial sale. Such containers may include injection or blow-molded plastic containers into which the desired vials are retained.

vi. Mass Spectrometry

By exploiting the intrinsic properties of mass and charge, mass spectrometry (MS) can resolved and confidently identified a wide variety of complex compounds, including proteins. Traditional quantitative MS has used electrospray ionization (ESI) followed by tandem MS (MS/MS) while newer quantitative methods are being developed using matrix assisted laser desorption/ionization (MALDI) followed by time of flight (TOF) MS. In particular, mass spectrometry has been applied to samples to identify proteins targets therein.

ESI is a convenient ionization technique that is used to produce gaseous ions from highly polar, mostly nonvolatile biomolecules, including lipids. The sample is injected as a liquid at low flow rates (1-10 μL/min) through a capillary tube to which a strong electric field is applied. The field generates additional charges to the liquid at the end of the capillary and produces a fine spray of highly charged droplets that are electrostatically attracted to the mass spectrometer inlet. The evaporation of the solvent from the surface of a droplet as it travels through the desolvation chamber increases its charge density substantially. When this increase exceeds the Rayleigh stability limit, ions are ejected and ready for MS analysis.

In ESI tandem mass spectroscopy (ESI/MS/MS), one is able to simultaneously analyze both precursor ions and product ions, thereby monitoring a single precursor product reaction and producing (through selective reaction monitoring (SRM)) a signal only when the desired precursor ion is present. When the internal standard is a stable isotope-labeled version of the analyte, this is known as quantification by the stable isotope dilution method. This approach has been used to accurately measure pharmaceuticals and bioactive peptides. Newer methods are performed on widely available MALDI-TOF instruments, which can resolve a wider mass range and have been used to quantify metabolites, peptides, and proteins. Larger molecules such as peptides can be quantified using unlabeled homologous peptides as long as their chemistry is similar to the analyte peptide. Protein quantification has been achieved by quantifying tryptic peptides. Complex mixtures such as crude extracts can be analyzed, but in some cases sample clean up is required.

Secondary ion mass spectroscopy, or SIMS, is an analytical method that uses ionized particles emitted from a surface for mass spectroscopy at a sensitivity of detection of a few parts per billion. The sample surface is bombarded by primary energetic particles, such as electrons, ions (e.g., O, Cs), neutrals or even photons, forcing atomic and molecular particles to be ejected from the surface, a process called sputtering. Since some of these sputtered particles carry a charge, a mass spectrometer can be used to measure their mass and charge. Continued sputtering permits measuring of the exposed elements as material is removed. This in turn permits one to construct elemental depth profiles. Although the majority of secondary ionized particles are electrons, it is the secondary ions which are detected and analysis by the mass spectrometer in this method.

Laser desorption mass spectroscopy (LD-MS) involves the use of a pulsed laser, which induces desorption of sample material from a sample site—effectively, this means vaporization of sample off of the sample substrate. This method is usually only used in conjunction with a mass spectrometer and can be performed simultaneously with ionization if one uses the right laser radiation wavelength.

When coupled with Time-of-Flight (TOF) measurement, LD-MS is referred to as LDLPMS (Laser Desorption Laser Photoionization Mass Spectroscopy). The LDLPMS method of analysis gives instantaneous volatilization of the sample, and this form of sample fragmentation permits rapid analysis without any wet extraction chemistry. The LDLPMS instrumentation provides a profile of the species present while the retention time is low and the sample size is small. In LDLPMS, an impactor strip is loaded into a vacuum chamber. The pulsed laser is fired upon a certain spot of the sample site, and species present are desorbed and ionized by the laser radiation. This ionization also causes the molecules to break up into smaller fragment-ions. The positive or negative ions made are then accelerated into the flight tube, being detected at the end by a microchannel plate detector. Signal intensity, or peak height, is measured as a function of travel time. The applied voltage and charge of the particular ion determines the kinetic energy, and separation of fragments is due to different size causing different velocity. Each ion mass will thus have a different flight-time to the detector.

One can either form positive ions or negative ions for analysis. Positive ions are made from regular direct photoionization, but negative ion formation requires a higher-powered laser and a secondary process to gain electrons. Most of the molecules that come off the sample site are neutrals, and thus can attract electrons based on their electron affinity. The negative ion formation process is less efficient than forming just positive ions. The sample constituents will also affect the outlook of a negative ion spectrum.

Other advantages with the LDLPMS method include the possibility of constructing the system to give a quiet baseline of the spectra because one can prevent coevolved neutrals from entering the flight tube by operating the instrument in a linear mode. Also, in environmental analysis, the salts in the air and as deposits will not interfere with the laser desorption and ionization. This instrumentation also is very sensitive, known to detect trace levels in natural samples without any prior extraction preparations.

Since its inception and commercial availability, the versatility of MALDI-TOF-MS has been demonstrated convincingly by its extensive use for qualitative analysis. For example, MALDI-TOF-MS has been employed for the characterization of synthetic polymers, peptide and protein analysis, DNA oligonucleotide sequencing, and the characterization of recombinant proteins. Recently, applications of MALDI-TOF-MS have been extended to include the direct analysis of biological tissues and single cell organisms with the aim of characterizing endogenous peptide and protein constituents.

The properties that make MALDI-TOF-MS a popular qualitative tool its ability to analyze molecules across an extensive mass range, high sensitivity, minimal sample preparation and rapid analysis times also make it a potentially useful quantitative tool. MALDI-TOF-MS also enables non-volatile and thermally labile molecules to be analyzed with relative ease. It is therefore prudent to explore the potential of MALDI-TOF-MS for quantitative analysis in clinical settings, for toxicological screenings, as well as for environmental analysis. In addition, the application of MALDI-TOF-MS to the quantification of peptides and proteins is particularly relevant. The ability to quantify intact proteins in biological tissue and fluids presents a particular challenge in the expanding area of proteomics and investigators urgently require methods to accurately measure the absolute quantity of proteins. While there have been reports of quantitative MALDI-TOF-MS applications, there are many problems inherent to the MALDI ionization process that have restricted its widespread use. These limitations primarily stem from factors such as the sample/matrix heterogeneity, which are believed to contribute to the large variability in observed signal intensities for analytes, the limited dynamic range due to detector saturation, and difficulties associated with coupling MALDI-TOF-MS to on-line separation techniques such as liquid chromatography. Combined, these factors are thought to compromise the accuracy, precision, and utility with which quantitative determinations can be made.

Because of these difficulties, practical examples of quantitative applications of MALDI-TOF-MS have been limited. Most of the studies to date have focused on the quantification of low mass analytes, in particular, alkaloids or active ingredients in agricultural or food products, whereas other studies have demonstrated the potential of MALDI-TOF-MS for the quantification of biologically relevant analytes such as neuropeptides, proteins, antibiotics, or various metabolites in biological tissue or fluid. In earlier work, it was shown that linear calibration curves could be generated by MALDI-TOF-MS provided that an appropriate internal standard was employed. This standard can “correct” for both sample-to-sample and shot-to-shot variability. Stable isotope labeled internal standards (isotopomers) give the best result.

With the marked improvement in resolution available on modern commercial instruments, primarily because of delayed extraction, the opportunity to extend quantitative work to other examples is now possible; not only of low mass analytes, but also biopolymers. Of particular interest is the prospect of absolute multi-component quantification in biological samples (e.g., proteomics applications).

The properties of the matrix material used in the MALDI method are critical. Only a select group of compounds is useful for the selective desorption of proteins and polypeptides. A review of all the matrix materials available for peptides and proteins shows that there are certain characteristics the compounds must share to be analytically useful. Despite its importance, very little is known about what makes a matrix material “successful” for MALDI. The few materials that do work well are used heavily by all MALDI practitioners and new molecules are constantly being evaluated as potential matrix candidates. With a few exceptions, most of the matrix materials used are solid organic acids. Liquid matrices have also been investigated but are not used routinely.

C. Combination Therapies

It may also be useful to treat cancers using the methods and compositions of the present disclosure, but further emply at least one other therapy. The present therapy and the other therapy would be provided in a combined amount effective to achieve a reduction in one or more disease parameter. This process may involve contacting the cells/subjects with the both agents/therapies at the same time, e.g., using a single composition or pharmacological formulation that includes both agents, or by contacting the cell/subject with two distinct compositions or formulations, at the same time, wherein one composition includes the compound and the other includes the other agent.

Alternatively, the antibody may precede or follow the other treatment by intervals ranging from minutes to weeks. One would generally ensure that a significant period of time did not expire between each delivery, such that the therapies would still be able to exert an advantageously combined effect on the cell/subject. In such instances, it is contemplated that one would contact the cell with both modalities within about 12-24 hours of each other, within about 6-12 hours of each other, or with a delay time of only about 12 hours. In some situations, it may be desirable to extend the time period for treatment significantly; however, where several days (2, 3, 4, 5, 6 or 7) to several weeks (1, 2, 3, 4, 5, 6, 7 or 8) lapse between the respective administrations.

It also is conceivable that more than one administration of either the compound or the other therapy will be desired. Various combinations may be employed, where the glutamate metabolism inhibitor of the present disclosure is “A,” and the other therapy is “B,” as exemplified below:

A/B/A B/A/B B/B/A A/A/B B/A/A A/B/B B/B/B/A B/B/A/B
A/A/B/B A/B/A/B A/B/B/A B/B/A/A B/A/B/A B/A/A/B B/B/B/A
A/A/A/B B/A/A/A A/B/A/A A/A/B/A A/B/B/B B/A/B/B B/B/A/B

Some agents or therapies suitable for use in a combined therapy with agents according to the present disclosure against cancer are discussed below, although other combinations are contemplated. The following is a general discussion of cancer therapies that may be used combination with the compounds of the present disclosure.

1. Chemotherapy

The term “chemotherapy” refers to the use of drugs to treat cancer. A “chemotherapeutic agent” is used to connote a compound or composition that is administered in the treatment of cancer. These agents or drugs are categorized by their mode of activity within a cell, for example, whether and at what stage they affect the cell cycle. Alternatively, an agent may be characterized based on its ability to directly cross-link DNA, to intercalate into DNA, or to induce chromosomal and mitotic aberrations by affecting nucleic acid synthesis. Most chemotherapeutic agents fall into the following categories: alkylating agents, antimetabolites, antitumor antibiotics, mitotic inhibitors, and nitrosoureas.

Examples of chemotherapeutic agents include alkylating agents such as thiotepa and cyclosphosphamide; alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethiylenethiophosphoramide and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard: nitrosureas such as carmustine, chlorozotocin, fotemustine, lomustine, nimustine, and ranimnustine; antibiotics such as the enediyne antibiotics (e.g., calicheamicin, especially calicheamicin gammaII and calicheamicin omegaII; dynemicin, including dynemicin A uncialamycin and derivatives thereof; bisphosphonates, such as clodronate; an esperamicin; as well as neocarzinostatin chromophore and related chromoprotein enediyne antiobiotic chromophores, aclacinomysins, actinomycin, authrarnycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalarnycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., paclitaxel and docetaxel; chlorambucil; gemcitabine; 6-thioguanine; mercaptopurine; methotrexate; platinum coordination complexes such as cisplatin, oxaliplatin and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluoromethylornithine (DMFO); retinoids such as retinoic acid; capecitabine; cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, paclitaxel, docetaxel, gemcitabien, navelbine, farnesyl-protein tansferase inhibitors, transplatinum, 5-fluorouracil, vincristin, vinblastin and methotrexate and pharmaceutically acceptable salts, acids or derivatives of any of the above.

2. Radiotherapy

Radiotherapy, also called radiation therapy, is the treatment of cancer and other diseases with ionizing radiation. Ionizing radiation deposits energy that injures or destroys cells in the area being treated by damaging their genetic material, making it impossible for these cells to continue to grow. Although radiation damages both cancer cells and normal cells, the latter are able to repair themselves and function properly.

Radiation therapy used according to the present disclosure may include, but is not limited to, the use of γ-rays, X-rays, and/or the directed delivery of radioisotopes to tumor cells. Other forms of DNA damaging factors are also contemplated such as microwaves and UV-irradiation. It is most likely that all of these factors induce a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells.

Radiotherapy may comprise the use of radiolabeled antibodies to deliver doses of radiation directly to the cancer site (radioimmunotherapy). Antibodies are highly specific proteins that are made by the body in response to the presence of antigens (substances recognized as foreign by the immune system). Some tumor cells contain specific antigens that trigger the production of tumor-specific antibodies. Large quantities of these antibodies can be made in the laboratory and attached to radioactive substances (a process known as radiolabeling). Once injected into the body, the antibodies actively seek out the cancer cells, which are destroyed by the cell-killing (cytotoxic) action of the radiation. This approach can minimize the risk of radiation damage to healthy cells.

Conformal radiotherapy uses the same radiotherapy machine, a linear accelerator, as the normal radiotherapy treatment but metal blocks are placed in the path of the x-ray beam to alter its shape to match that of the cancer. This ensures that a higher radiation dose is given to the tumor. Healthy surrounding cells and nearby structures receive a lower dose of radiation, so the possibility of side effects is reduced. A device called a multi-leaf collimator has been developed and may be used as an alternative to the metal blocks. The multi-leaf collimator consists of a number of metal sheets which are fixed to the linear accelerator. Each layer can be adjusted so that the radiotherapy beams can be shaped to the treatment area without the need for metal blocks. Precise positioning of the radiotherapy machine is very important for conformal radiotherapy treatment and a special scanning machine may be used to check the position of internal organs at the beginning of each treatment.

High-resolution intensity modulated radiotherapy also uses a multi-leaf collimator. During this treatment the layers of the multi-leaf collimator are moved while the treatment is being given. This method is likely to achieve even more precise shaping of the treatment beams and allows the dose of radiotherapy to be constant over the whole treatment area.

Although research studies have shown that conformal radiotherapy and intensity modulated radiotherapy may reduce the side effects of radiotherapy treatment, it is possible that shaping the treatment area so precisely could stop microscopic cancer cells just outside the treatment area being destroyed. This means that the risk of the cancer coming back in the future may be higher with these specialized radiotherapy techniques.

Scientists also are looking for ways to increase the effectiveness of radiation therapy. Two types of investigational drugs are being studied for their effect on cells undergoing radiation. Radiosensitizers make the tumor cells more likely to be damaged, and radioprotectors protect normal tissues from the effects of radiation. Hyperthermia, the use of heat, is also being studied for its effectiveness in sensitizing tissue to radiation.

3. Immunotherapy

In the context of cancer treatment, immunotherapeutics, generally, rely on the use of immune effector cells and molecules to target and destroy cancer cells. Trastuzumab (Herceptin™) is such an example. The immune effector may be, for example, an antibody specific for some marker on the surface of a tumor cell. The antibody alone may serve as an effector of therapy or it may recruit other cells to actually affect cell killing. The anti body also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., direct cytotoxic activity and inhibition or reduction of ErbB2 Would provide therapeutic benefit in the treatment of ErbB2 overexpressing cancers.

In one aspect of immunotherapy, the tumor cell must bear some marker that is amenable to targeting, i.e., is not present on the majority of other cells. Many tumor markers exist and any of these may be suitable for targeting in the context of the present disclosure. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, M4ucB, PLAP, estrogen receptor, laminin receptor, erb B and p155. An alternative aspect of immunotherapy is to combine anticancer effects with immune stimulatory effects. Immune stimulating molecules also exist including cytokines such as IL-2, IL-4, IL-12, GM-CSF, γ-IFN, chemokines such as MIP-1, MCP-1, IL-8, and growth factors such as FLT3 ligand. Combining immune stimulating molecules, either as proteins or using gene delivery in combination with a tumor suppressor has been shown to enhance anti-tumor effects (Ju et al., 2000). Moreover, antibodies against any of these compounds may be used to target the anti-cancer agents discussed herein.

Examples of immunotherapies currently under investigation or in use are immune adjuvants e.g., Mycobacterium bovis, Plasmodium falciparum, dinitrochlorobenzene and aromatic compounds (U.S. Pat. Nos. 5,801,005 and 5,739,169; Hui and Hashimoto, 1998; Christodoulides et al., 1998), cytokine therapy, e.g., interferons α, β, and γ; IL-1, GM-CSF and TNF (Bukowski et al., 1998; Davidson et al., 1998; Hellstrand et al., 1998) gene therapy, e.g., TNF, IL-1, IL-2, p53 (Qin et al., 1998; Austin-Ward and Villaseca, 1998; U.S. Pat. Nos. 5,830,880 and 5,846,945) and monoclonal antibodies. e.g., anti-ganglioside GM2, anti-HER-2, anti-p185 (Pietras et al., 1998; Hanibuchi et al., 1998; U.S. Pat. No. 5,824,311).

In active immunotherapy, an antigenic peptide, polypeptide or protein, or an autologous or allogenic tumor cell composition or “vaccine” is administered, generally with a distinct bacterial adjuvant (Ravindranath and Morton, 1991; Morton et al., 1992; Mitchell et al., 1990; Mitchell et al., 1993).

In adoptive immunotherapy, the patient's circulating lymphocytes, or tumor infiltrated lymphocytes, are isolated in vitro, activated by lymphokines such as IL-2 or transduced with genes for tumor necrosis, and readministered (Rosenberg et al., 1988; 1989).

Checkpoint inhibitor therapy is a form of cancer treatment immunotherapy currently under research. The therapy targets immune checkpoints, key regulators of the immune system that stimulate or inhibit its actions, which tumors can use to protect themselves from attacks by the immune system. Checkpoint therapy can block inhibitory checkpoints, restoring immune system function. The first anti-cancer drug targeting an immune checkpoint was ipilimumab, a CTLA-4 blocker approved in the United States in 2011.

Currently approved checkpoint inhibitors target the molecules CTLA4, PD-1, and PD-L1. PD-1 is the transmembrane programmed cell death 1 protein (also called PDCD1 and CD279), which interacts with PD-L1 (PD-1 ligand 1, or CD274). PD-L1 on the cell surface binds to PD1 on an immune cell surface, which inhibits immune cell activity. Among PD-L1 functions is a key regulatory role on T cell activities. It appears that (cancer-mediated) upregulation of PD-L1 on the cell surface may inhibit T cells that might otherwise attack. Antibodies that bind to either PD-1 or PD-L1 and therefore block the interaction may allow the T-cells to attack the tumor.

In some embodiments, the immune checkpoint inhibitor therapy may be molecules targeting adenosine A2A receptor (A2AR), B7-H3 (also known as CD276), B and T lymphocyte attenuator (BTLA), cytotoxic T-lymphocyte-associated protein 4 (CTLA-4, also known as CD152), indoleamine 2,3-dioxygenase (IDO), killer-cell immunoglobulin (KIR), lymphocyte activation gene-3 (LAG3), T-cell immunoglobulin domain and mucin domain 3 (TIM-3) and V-domain Ig suppressor of T cell activation (VISTA).

The immune checkpoint inhibitors may be drugs such as small molecules, recombinant forms of ligand or receptors, or, in particular, are antibodies, such as human antibodies (e.g., International Patent Publication No. WO2015016718; both incorporated herein by reference). Known inhibitors of the immune checkpoint proteins or analogs thereof may be used, in particular chimerized, humanized or human forms of antibodies may be used. As the skilled person will know, alternative and/or equivalent names may be in use for certain antibodies mentioned in the present disclosure. Such alternative and/or equivalent names are interchangeable in the context of the present disclosure. For example, it is known that lambrolizumab is also known under the alternative and equivalent names MK-3475 and pembrolizumab.

4. Surgery

Approximately 60% of persons with cancer will undergo surgery of some type, which includes preventative, diagnostic or staging, curative, and palliative surgery. Curative surgery is a cancer treatment that may be used in conjunction with other therapies, such as the treatment of the present disclosure, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and microscopically controlled surgery (Mohs' surgery). It is further contemplated that the present disclosure may be used in conjunction with removal of superficial cancers, precancers, or incidental amounts of normal tissue.

Upon excision of part or all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

In some particular embodiments, after removal of the tumor, an adjuvant treatment with a compound of the present disclosure is believed to be particularly efficacious in reducing the reoccurrence of the tumor. Additionally, the compounds of the present disclosure can also be used in a neoadjuvant setting.

It also should be pointed out that any of the foregoing therapies may prove useful by themselves in treating cancer.

5. Metformin

Another possible combination with the inhibitors of the present disclosure is Metformin, which is marketed under the trade name Glucophage among others (Glucophage XR, Carbophage SR, Riomet, Fortamet, Glumetza, Obimet, Gluformin, Dianben, Diabex, Diaformin, Siofor, Metfogamma and Glifor). Metformin may also reduce the insulin requirement in type 1 diabetes, albeit with an increased risk of hypoglycemia.

Liquid metformin is sold under the name Riomet in India. Each 5 ml of Riomet is equivalent to the 500-mg tablet form. Metformin IR (immediate release) is available in 500, 850, and 1000-mg tablets. All of these are available as generic medications in the U.S. Metformin SR (slow release) or XR (extended release) was introduced in 2004. It is available in 500, 750, and 1000-mg strengths, mainly to counteract common gastrointestinal side effects, as well as to increase compliance by reducing pill burden. No difference in effectiveness exists between the two preparations. This molecule is the first-line medication for the treatment of type 2 diabetes, particularly in people who are overweight. It is also used in the treatment of polycystic ovary syndrome. Metformin may also reduce the insulin requirement in type 1 diabetes, albeit with an increased risk of hypoglycemia.

Metformin is taken by mouth and is generally well-tolerated. Common side effects include diarrhea, nausea, and abdominal pain. It has a low risk of causing low blood sugar. High blood lactic acid level is a concern if the medication is prescribed inappropriately or in overly large doses. It should not be used in those with significant liver disease or kidney problems. While no clear harm comes from use during pregnancy, insulin is generally preferred for gestational diabetes. Metformin is a biguanide antihyperglycemic agent. It works by decreasing glucose production by the liver and increasing the insulin sensitivity of body tissues.

Metformin has little or no effect on body weight in type 2 diabetes compared with placebo, in contrast to sulfonylureas which are associated with weight gain. There is some evidence that metformin is associated with weight loss in obesity in the absence of diabetes. Metformin has a lower risk of hypoglycemia than the sulfonylureas, although hypoglycemia has uncommonly occurred during intense exercise, calorie deficit, or when used with other agents to lower blood glucose. Metformin modestly reduces LDL and triglyceride levels.

In those with polycystic ovarian syndrome (PCOS), tentative evidence shows that metformin use increases the rate of live births. This includes in those who have not been able to get pregnant with clomiphene. Metformin does not appear to change the risk of miscarriage. A number of other benefits have also been found both during pregnancy and in nonpregnant people with PCOS. In women with PC(OS undergoing in vitro fertilization, evidence does not support a benefit with respect to live births. The evidence does not support general use during pregnancy for improving maternal and infant outcomes in obese women.

A review of metformin use during pregnancy compared to insulin alone found good short-term safety for both the mother and baby, but unclear long-term safety. Several observational studies and randomized, controlled trials found metformin to be as effective and safe as insulin for the management of gestational diabetes. Nonetheless, several concerns have been raised and evidence on the long-term safety of metformin for both mother and child is lacking. Compared with insulin, women with gestational diabetes treated with metformin gain less weight and are less likely to develop pre-eclampsia during pregnancy. Babies born to women treated with metformin have less visceral fat, and this may make them less prone to insulin resistance in later life.

Metformin appears to be safe and effective in counteracting the weight gain caused by the antipsychotic medications olanzapine and clozapine. Although modest reversal of clozapine-associated weight gain is found with metformin, primary prevention of weight gain is more valuable.

The FDA most recently revised its prescribing information on metformin in 2016. Current advice is that metformin is contraindicated in people with 1) severe renal impairment (estimated glomerular filtration rate (eGFR) below 30 ml/min/1.73 m2); 2) known hypersensitivity to metformin; or 3) acute or chronic metabolic acidosis, including diabetic ketoacidosis, with or without coma. Warnings are also given regarding the use of metformin in less severe renal impairment, people aged 65 years old or greater, hypoxic states (e.g., acute congestive heart failure), excessive alcohol intake, hepatic impairment, concomitant use of certain drugs (e.g., carbonic anhydrase inhibitors such as topiramate), surgery, and other procedures, or in people having a radiological study with administration of an iodinated contrast agent.

Metformin is recommended to be temporarily discontinued before any procedure involving use of iodinated contrast agents, (such as a contrast-enhanced CT scan or angiogram) due to the increased risk of lactic acidosis resulting from impaired renal function; metformin can be resumed after two days after contrast administration, if renal function is adequate and stable.

The most common adverse effect of metformin is gastrointestinal irritation, including diarrhea, cramps, nausea, vomiting, and increased flatulence; metformin is more commonly associated with gastrointestinal side effects than most other antidiabetic medications. The most serious potential side effect of metformin use is lactic acidosis; this complication is very rare, and the vast majority of these cases seem to be related to comorbid conditions, such as impaired liver or kidney function, rather than to the metformin itself.

The H₂-receptor antagonist cimetidine causes an increase in the plasma concentration of metformin by reducing clearance of metformin by the kidneys; both metformin and cimetidine are cleared from the body by tubular secretion, and both, particularly the cationic (positively charged) form of cimetidine, may compete for the same transport mechanism. A small double-blind, randomized study found the antibiotic cephalexin to also increase concentrations by a similar mechanism; theoretically, other cationic medications may produce the same effect.

Metformin also interacts with anticholinergic medications, due to their effect on gastric motility. Anticholinergic drugs reduce gastric motility, prolonging the time drugs spend in the gastrointestinal tract. This impairment may lead to more being absorbed than without the presence of an anticholinergic drug, thereby increasing the concentration of in the plasma and increasing the risk for adverse effects.

The molecular mechanism of is incompletely understood. Multiple potential mechanisms of action have been proposed: inhibition of the mitochondrial respiratory chain (complex I), activation of AMP-activated protein kinase (AMPK), inhibition of glucagon-induced elevation of cyclic adenosine monophosphate (cAMP) with reduced activation of protein kinase A (PKA), inhibition of mitochondrial glycerophosphate dehydrogenase, and an effect on gut microbiota. Ultimately, it decreases gluconeogenesis (liver glucose production). It also has an insulin-sensitizing effect with multiple actions on tissues including the liver, skeletal muscle, endothelium, adipose tissue, and the ovary. The average patient with type 2 diabetes has three times the normal rate of gluconeogenesis; treatment reduces this by over one-third.

### V. EXAMPLES

The following examples are included to demonstrate preferred embodiments. It should be appreciated by those of skill in the art that the techniques disclosed in the examples that follow represent techniques discovered by the inventor to function well in the practice of embodiments, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1

For Arid1a−/−/Pik3caH1047R genetic ovarian clear cell ovarian carcinoma mouse model, intrabursal adenovirus-Cre injection was used to induce ovarian clear cell carcinoma formation in 6-8 week-old female mice. Mice were randomized into six groups four weeks after injection. The mice were randomized into the following four treatment groups: vehicle and IgG control, CB-839 (200 mg/kg twice daily, orally) and IgG control, vehicle control and anti-PDL1 (10 mg/kg, twice a week, i.p.), and a combination of CB-839 and anti-PDL1. At the end of treatments, mice were euthanized and tumors were surgically dissected. Tumor burden was calculated on the basis of tumor weight. The survival experiment was performed following The Wistar Institute IACUC guideline (tumor burden exceeds 10% of body weight).

Immune cell profiling was analyzed as previously described (Fukimoto et al., 2019). Briefly, tumor cells were extracted using Mouse Dissociation Kit (Miltenyi Biotec, cat. no. 130-096-730) according to the manufacture's instructions. The cells were then mashed with 70-μM strainer and used for staining. For peritoneal wash, peritoneal cavity of mice was washed three times with 5 ml PBS and incubated in RBC lysis buffer (Thermo Fisher, cat. no. 00-4333-57). Live/dead cells were discriminated by Zombie Yellow™ Fixable Viability Kit (Biolegend, cat. no. 423103). Cell surface staining was performed using antibodies against CD3e (BD, cat. no. 552774), CD45 (Biolegend, cat. no. 103147), CD4 (Biolegend, cat. no. 100516), CD8a (Biolegend, cat. no. 100708), CD69 (Biolegend, cat. no. 104510), PD1 (Biolegend, cat. no. 109109) and PDL1 (Biolegend, cat. no. 124321). Data were acquired using LSRII-18 and analyzed using FlowJo software.

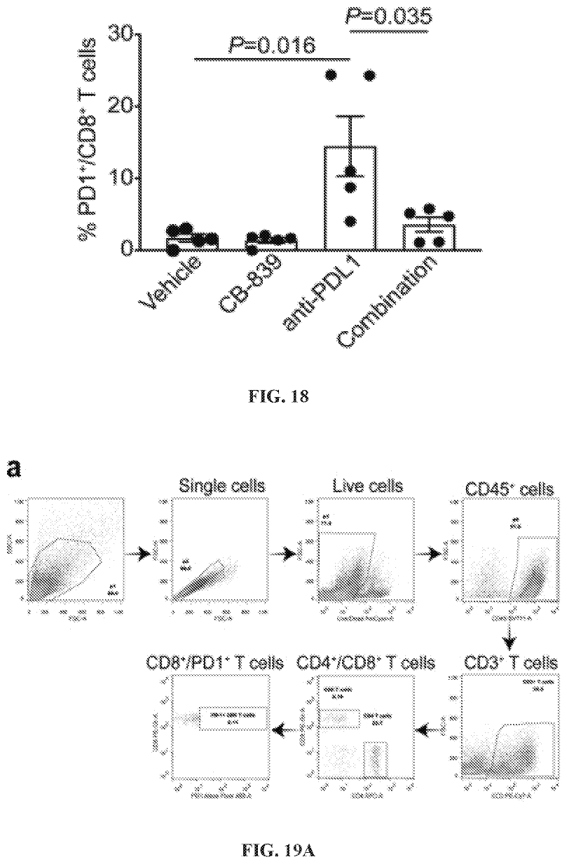

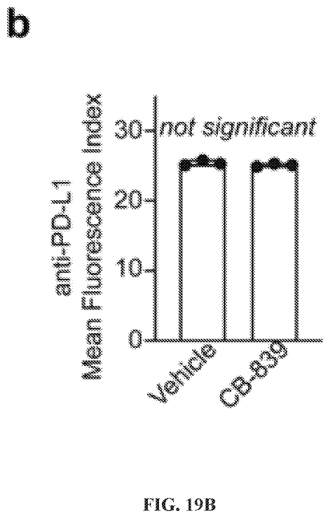

Consistent with previous reports, CB-839 treatment prevented CD8 T cell exhaustion induced by anti-PDL1 antibody as evidenced by a decrease in PD-1 position CD8 T cells (FIG. 18 and FIG. 19A). Notably, CB-839 did not affect PDL1 expression on ARID1A-mutated TOV21G cells (FIG. 19B).

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the compositions and methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### VI. REFERENCES

The following references, to the extent that they provide exemplary procedural or other details supplementary to those set forth herein, are specifically incorporated herein by reference.
U.S. Pat. No. 3,817,837
U.S. Pat. No. 3,850,752
U.S. Pat. No. 3,939,350
U.S. Pat. No. 3,996,345
U.S. Pat. No. 4,275,149
U.S. Pat. No. 4,277,437
U.S. Pat. No. 4,366,241
U.S. Pat. No. 5,538,880
U.S. Pat. No. 5,928,906
“Antibodies: A Laboratory Manual,” Cold Spring Harbor Press, Cold Spring Harbor, N.Y., 1988.
Abbondanzo et al., Am. J. Pediatr. Hematol. Oncol., 12(4), 480-489, 1990.
Allred et al., Arch. Surg., 125(1), 107-113, 1990.
Ausubel et al., Current Protocols in Molecular Biology, Greene Publishing Associates and Wiley Interscience, N.Y., 1994.
Brown et al., J. Immunol. Meth., 12; 130(1):111-121, 1990.
Campbell, In: Monoclonal Antibody Technology, Laboratory Techniques in Biochemistry and Molecular Biology, Vol. 13, Burden and Von Knippenberg, Eds. pp. 75-83, Amsterdam, Elsevier, 1984.
De Jager et al., Semin. Nucl. Med. 23(2), 165-179, 1993.
Doolittle and Ben-Zeev, Methods Mol. Biol., 109, 215-237, 1999.
Fukumoto et al., Cancer Res, doi:10.1158/0008-5472.CAN-19-1302, 2019.
Goding, In: Monoclonal Antibodies: Principles and Practice, 2d ed., Orlando, Fla., Academic Press, 60-61, 65-66, 71-74, 1986.
Gulbis and Galand, Hum. Pathol. 24(12), 1271-1285, 199³.
Kohler and Milstein, Eur. Immunol., 6, 511-51⁹, 1976.
Kohler and Milstein, Nature, 256, 495-497, 1975.
Nakamura et al., In: Enzyme Immunoassays: Heterogeneous and Homogeneous Systems, Chapter 27, 1987.
Remington's Pharmaceutical Sciences, 15^{th} Ed., 33:624-652, 1990.
Sambrook et al, In: Molecular cloning: a laboratory manual, 2^{nd}Ed., Cold Spring Harbor Laboratory Press, Cold Spring Harbor, N.Y., 1989.

1. A method of treating a subject determined to have an ARID1A-mutated cancer, pre-cancer or benign tumor comprising administering to said subject at least one inhibitor of glutamate metabolism.

2. The method of claim 1, wherein the at least one inhibitor of glutamate metabolism is telaglenastat, diazooxonorleucine, OP-329 and/or OP-330.

3. The method of claim 1, further comprising treating said subject with a second cancer therapy.

4. The method of claim 3, wherein said second cancer therapy is an inhibitor of aspartate biosynthesis.

5. The method of claim 4, wherein said inhibitor of aspartate biosynthesis is metformin.

6. The method of claim 3, wherein said second cancer therapy is chemotherapy, radiotherapy, immunotherapy (e.g., checkpoint inhibitor), hormonal therapy, toxin therapy or surgery.

7. The method of claim 1, further comprising determining, prior to treating, that said subject has an ARID1A-mutated cancer, pre-cancer or benign tumor.

8. The method of claim 7, wherein determining comprises:
(a) obtaining a sample from said subject that contains protein and/or nucleics acids; and
(b) determining mutation status of an ARID1A protein or nucleic acid encoding ARID1A comprising the sequence of SEQ ID NO: 1 in said sample.

9. The method of claim 8, wherein determining comprises a nucleic acid-based assay.

10. The method of claim 8, wherein determining comprises a protein-based assay.

11. The method of claim 8, wherein said biological sample is a fluid sample.

12. The method of claim 11, wherein said fluid sample is blood, serum plasma, sputum, saliva, urine or nipple aspirate.

13. The method of claim 8, wherein said biological sample is a tissue sample.

14. The method of claim 13, wherein said tissue sample is a cancer, pre-cancer or benign tumor tissue sample.

15. The method of claim 1, wherein said cancer is breast cancer, pancreatic cancer, gastric cancer, or ovarian cancer, such as ovarian clear cell carcinoma.

16. The method of claim 1, wherein said subject is a human subject.

17. The method of claim 1, wherein said subject is a non-human primate.

18. The method of claim 1, wherein said subject has previously been diagnosed with cancer, a pre-cancer or a benign tumor.

19. The method of claim 1, wherein said cancer is recurrent, primary, metastatic or multi-drug resistant.

20. The method of claim 1, wherein said inhibitor of glutamate metabolism is administered more than once, such as daily, every other day, weekly, monthly and/or on a chronic basis.

21. The method of claim 6, wherein said checkpoint inhibitor is a PD-1 inhibitor, a PD-L1 inhibitor or a CTLA-4 inhibitor.

22. The method of claim 21, wherein said checkpoint inhibitor is an anti-PD-1 antibody (e.g., pembrolizumab; nivolumab, cemiplimab), an anti-PD-L1 antibody (e.g., atezolizumab, avelumab, durvalumab) or an anti-CTLA-4 antibody (ipilumumab).
